(19) United States
(12) Patent Application Publication
(10) Pub. No.: US 2003/0091592 A1
(43) Pub. Date: May 15, 2003

(54) Generation of virus-like particles by VSV

(76) Inventor: Glen N. Barber, Miami, FL (US)

Correspondence Address: Debra J. Glaister, Morrison & Foerster LLP, 755 Page Mill Road, Palo Alto, CA, 94304-1018, US

(21) Appl. No.: 10/268,133

(22) Filed: Oct. 9, 2002

Related U.S. Application Data
(60) Provisional application No. 60/327,296, filed on Oct. 9, 2001.
(60) Provisional application No. 60/327,295, filed on Oct. 9, 2001.

(51) Int. Cl. C12Q 1/70; C12Q 1/68; A61K 39/21; A61K 39/12

(52) U.S. Cl. 424/199.1; 424/208.1; 424/207.1; 424/188.1; 435/6; 435/5

(57) ABSTRACT

The present invention provides VSV vectors comprising nucleic acid encoding a HTLV-1 viral protein, such as HTLV-1 gag and env proteins, from any strain of HTLV-1 for the production of HTLV-1 VLPs. The present invention provides VSV vectors comprising nucleic acid encoding a HPV viral protein, such as HPV L1 or L1 and L2, from any strain of HPV for the production of HPV VLPs. The present invention also provides methods of making such vectors, host cells, expression systems, and compositions comprising such VSV vectors, and viral particles comprising such VSV vectors. The present invention also provides vaccine compositions and provides methods for eliciting an immune response in an individual and methods for ameliorating symptoms of disease.

### CROSS-REFERENCE TO RELATED APPLICATIONS

The present application claims the benefit of priority to U.S. provisional application 60/327,296 filed Oct. 9, 2001 and provisional application 60/327,295 filed Oct. 9, 2001 which are hereby incorporated herein in their entirety by reference.

### STATEMENT REGARDING FEDERALLY SPONSORED RESEARCH OR DEVELOPMENT

NOT APPLICABLE

### FIELD OF INVENTION

The present invention generally relates to vesicular stomatitis virus (VSV), and to the use of recombinant VSV for generating virus-like particles, including HTLV-1 virus like particles and HPV virus like particles.

### BACKGROUND OF THE INVENTION

Vesicular stomatitis virus (VSV), of the genus, Vesiculovirus, is the prototypic member of the family Rhabdoviridae, and is an enveloped virus with a negative stranded RNA genome that causes a self-limiting disease in live-stock and is essentially non-pathogenic in humans. Balachandran and Barber (2000, IUBMB Life 50: 135-8). Rhabdoviruses have single, negative-strand RNA genomes of 11,000 to 12,000 nucleotides (Rose and Schubert, 1987, Rhabdovirus genomes and their products, in The Viruses: The Rhabdoviruses, Plenum Publishing Corp., NY, pp. 129-166). The virus particles contain a helical, nucleocapsid core composed of the genomic RNA and protein. Generally, three proteins, termed N (nucleocapsid, which encases the genome tightly), P (formerly termed NS, originally indicating nonstructural), and L (large) are found to be associated with the nucleocapsid. An additional matrix (M) protein lies within the membrane envelope, perhaps interacting both with the membrane and the nucleocapsid core. A single glycoprotein (G) species spans the membrane and forms the spikes on the surface of the virus particle. Glycoprotein G is responsible for binding to cells and membrane fusion. The VSV genome is the negative sense (i.e., complementary to the RNA sequence (positive sense) that functions as mRNA to directly produce encoded protein), and rhabdoviruses must encode and package an RNA-dependent RNA polymerase in the virion (Baltimore et al., 1970, Proc. Natl. Acad. Sci. USA 66: 572-576), composed of the P and L proteins. This enzyme transcribes genomic RNA to make subgenomic mRNAs encoding the 5-6 viral proteins and also replicates full-length positive and negative sense RNAs. The genes are transcribed sequentially, starting at the 3′ end of the genomes.

The sequences of the VSV mRNAs and genome is described in Gallione et al. 1981, J Virol. 39:529-535; Rose and Gallione, 1981, J Virol. 39:519-528; Rose and Schubert, 1987, Rhabdovirus genomes and their products, p.129-166, in R. R. Wagner (ed.), The Rhabdoviruses. Plenum Publishing Corp., NY; Schubert et al., 1985, Proc. Natl. Acad. Sci. USA 82:7984-7988. WO 96/34625 published Nov. 7, 1996, disclose methods for the production and recovery of replicable vesiculovirus. U.S. Pat. No. 6,168,943, issued Jan. 2, 2001, describes methods for making recombinant vesiculoviruses.

Expression of HPV L1 or L1 and L2 in bacteria, yeast, or eukaryotic cells is disclosed in Chen et al. (2001, J. Mol. Bio. Vol. 307:173-82); Rossi et al. (2000, Hum. Gene Ther., vol. 11 1165-1176); Zhou et al., 1991, Virology, vol. 185:251-7; Reuter et al. (2002, J. Virol. Vol. 76:8900-8909) and Fang et al. (2000, Biotechnol. Appl. Biochem. vol. 32(pt 1):27-33). HTLV-1 virus like particles are disclosed in Bouamr, et al. (2000, Virology, vol. 278:597-609).

### BRIEF SUMMARY OF THE INVENTION

The present invention provides recombinant vesicular stomatitis virus (VSV) vectors comprising isolated nucleic acid encoding part or all of a HTLV-1 Gag gene and part or all of a viral Env gene, wherein said part or all of said HTLV-1 Gag gene and said part or all of the viral Env gene are capable of assembling into a HTLV-1 virus like particle. In additional examples, the VSV vector further comprises HTLV-1 pro function. In some examples, the part of the Gag gene is selected from the group consisting of p19, p24 and p15. In other examples, the viral Env gene is a HTLV-1 Env gene. In some examples, the VSV vector is replication-competent and in other examples, is replication-defective. In further examples, the VSV vector comprises a deletion of the G-protein function. In yet other examples, the VSV vector may further comprise additional HTLV-1 viral proteins.

The present invention also provides HTLV-1 virus like particles comprising part or all of a HTLV-1 Gag gene and part or all of a viral Env gene. In some examples, the part or all of the viral Env gene is a HTLV-1 Env gene.

The present invention also provides methods of producing a HTLV-1 virus like particle (VLP) comprising growing a cell comprising a VSV vector comprising isolated nucleic acid encoding part or all of a HTLV-1 Gag gene and part or all of a viral Env gene under conditions suitable for expression of the HTLV-1 Gag and viral Env and assembly into a VLP, and optionally isolating said VLP. The present invention also provides methods of producing a HTLV-1 VLP comprising growing a cell expressing viral env function and comprising a VSV vector comprising part or all of a HTLV-1 Gag gene under conditions suitable for expression of the HTLV-1 Gag and viral Env and assembly into a VLP, and optionally isolating said VLP. The present invention also provides methods of producing a HTLV-1 VLP comprising growing a cell expressing HTLV-1 gag function and comprising a VSV vector comprising part or all of a viral env gene under conditions suitable for expression of the HTLV-1 Gag and viral Env and assembly into a VLP and optionally isolating said VLP. In some examples of the methods the viral env is HTLV-1 env. In some examples, the cell is mammalian cell. In other examples, the VSV is replication competent and in other examples is replication-defective. In yet other examples, the VSV vector lacks G-protein function. In additional examples, the invention provides HTLV-1 virus like particles made by the methods. The present invention also encompasses methods of eliciting an immune response in an individual comprising administering to the individual a HTLV-1 virus like particle. The invention also provides methods of ameliorating symptoms of a disease comprising administering to the individual a HTLV-1 virus like particle. The present invention also encompasses cells and compositions, including vaccine compositions, comprising VSV vectors comprising HTLV-1 protein(s), VSV viral particles comprising HTLV-1 protein(s), and/or HTLV-1 virus like particles. In some examples, the HTLV-1 virus like particle is present in the composition in an amount effective to elicit an immune response in an individual. In other examples, compositions further comprise a pharmaceutically acceptable excipient.

The present invention also encompasses recombinant vesicular stomatitis virus (VSV) vector comprising isolated nucleic acid encoding a HPV L1 protein wherein said HPV L1 protein is capable of assembling into a HPV virus like particle. In some examples, the VSV vector further comprises nucleic acid encoding HPV L2 protein. In other examples, the HPV is any strain of HPV including but not limited to HPV strain 16, 18, 31, 33 and 45. In further examples, the VSV vector is replication competent and yet other examples is replication-defective. In additional examples, the VSV vector lacks G-protein function. The present invention also provides methods of producing a HPV virus like particle (VLP) comprising growing a cell comprising a VSV vector comprising nucleic acid encoding a HPV L1 or L1 and L2 protein under conditions suitable for expression of HPV L1 or L1 and L2 protein and assembly into a HPV VLP, and optionally isolating said VLP. In some examples, the cell is mammalian cell. In other examples of the methods the VSV is replication competent. In yet other examples, the VSV is replication-defective. In further examples, the VSV vector lacks G-protein function. The present invention also provides HPV virus like particles made by the methods. The present invention also provides methods of eliciting an immune response in an individual comprising administering to the individual a VSV produced HPV virus like particle. The invention also provides methods of ameliorating symptoms of disease comprising administering to the individual a VSV produced HPV virus like particle. The present invention also encompasses cells and compositions, including vaccine compositions, comprising VSV vectors comprising HPV protein(s), VSV viral particles comprising HPV protein(s), and/or HPV virus like particles. In some examples, the HPV virus like particle is present in the composition in an amount effective to elicit an immune response in an individual. In other examples, compositions further comprise a pharmaceutically acceptable excipient.

### BRIEF DESCRIPTION OF THE SEVERAL VIEWS OF THE DRAWING(S)

FIGS. 1A-1B. (A) Construction of rVSV expressing HPV L1. The L1 region of the HPV polypeptide was cloned into the XhoI and NheI sites of the rVSV replicon vector pVSV-XN2 by PCR. (B) Growth analysis of recombinant viruses. VSV-HPV-L1 demonstrates a similar growth rate to rVSV-GFP. BHK cells were infected at an m.o.i. of 10. Cell medium was collected at 6, 12, 18, and 24 h post-infection and virus titers determined by plaque assay as described Balachandran et al. (2000, J. Virol., vol 74:1513-23).

FIGS. 2A-2C. Expression of HPV L1. BHK cells were infected with VSV-HPV-L1, wild type VSV or control virus VSV-XN2 at an m.o.i. of 1 (FIG. 2A). After 18 hours, cells were lysed and HPV protein expression determined by immunoblot analysis (FIG. 2B). VSV proteins were detected by polyclonal mouse antiserum generated in Balb/c mice infected with VSV. Immunofluorescence analysis of HPV structural proteins. Expression and intracellular localization of HPV structural proteins was confirmed by immunofluorescence using monoclonal antibody specific for L1 (FIG. 2C).

FIGS. 3A-3B. (A ) HPV-L1 can be detected in the cell medium. immunoblot analysis for L1 protein in cell medium from VSV-HPV-L1 or control virus infected cells (concentrated by ultracentrifugation). (B) Gradient purified HPV L1 forms macromolecular complexes. CsCl gradient fractions containing HPV-LPs were analyzed for L1 protein by immunoblot. L1 could be detected predominantly in fraction 16, while VSV proteins were found throughout fractions 14-24.

FIG. 4. Electron microscopy images of partially purified HPV-LPs. HPV-like particles were adsorbed to carbon coated copper grids and then negative stained with 2% uranyl acetate for 2-3 min and then visualized by transmission electron microscopy.

FIGS. 5A-5B. FIG. 5A Schematic representation of the VSV genome showing sites of insertion of the HTLV-1 gag-pro and env genes. FIG. 5B. Growth curves of recombinant viruses. BHK cells were infected with VSV-GFP or VSV-HTLV-1 gag-env at an m.o.i. of 10. Supernatants from infected cells were harvested at the indicated time points postinfection, and viral titers were determined by plaque assay.

FIGS. 6A-6B. Expression of HTLV-1gag-pro and env genes in BHK cells infected with VSV-gag-pro-env. BHK cells were infected with VSV-gag-pro-env or rVSV at an m.o.i. of 1 for 24 hours. Cell lysates were analyzed for gag and env expression with (FIG. 6A) anti-p24 of (FIG. 6B) anti-env antibodies.

FIGS. 7A-7B. Immunoflourescence of HTLV-1 env expression from VSV-HTLV-1gag-env. BHK cells were infected with (FIG. 7B) wild type VSV or (FIG. 7A) VSV-gag-env at an m.o.i. of 1 for 16 hours, fixed and stained with anti-env antibody followed by a FITC-conjugated goat anti-mouse (1:100; Gibco-BRL;) in 0.1% Brij-97/PBS for 1 hour at 4° C.

FIG. 8. HTLV-1 envelope and processed gag proteins are released into the cell medium as VLPs. HTLV-1 Env and the fully processed gag protein (p24 and p19) were centrifuged and detected by immunoblot analysis from the cell medium of VSV-HTLV1-gag/env but not VSV infected BHK cells.

FIG. 9A-9B. FIG. 9A. Mouse dendritic cells (DC) were infected for 1 hr with VSV virus coding for the GFP protein at different m.o.i. (left panel, 0.1 m.o.i.; middle panel, 1.0 m.o.i; and right panel, 10 m.o.i.) and cells analyzed by FACS 24 hours later. The percentage of live and GFP+ cells is shown in the lower right quadrant. Lower panel represents FACS analysis of DC cultured in the presence of anti mouse IFN α/β antibodies (1000 U/ml) that were added to DC culture following virus infection. Upper panel represent DC cultures without addition of neutralizing IFN antibodies. FIG. 9B. Quantification of the transduction rate of VSV GFP by DC. Replication of VSV was assessed by measuring the percentage of GFP positive cells by flow cytometry.

FIG. 10. Production of infectious VSV particles by mouse DC. DC were infected with live VSV virus at different m.o.i. for 1 hr. Non bound virus was removed by extensive washing and virus release into the supernatants was determined after 24 and 48 hours.

FIGS. 11A-11B. Human, monocyte derived, DC were infected at different m.o.i. (left panel, 0.1 m.o.i.; middle panel, 1.0 m.o.i; and right panel, 10 m.o.i.) with GFP encoding recombinant VSV virus for 1 hour. DC were cultured for 24 h following VSV infection in DC media with (FIG. 11B) or without (FIG. 11A) LPS added (1 μg/ml) and the expression of GFP quantified by flow cytometry. Percentages of cells staining positive for GFP are indicated.

FIGS. 12A-12D. Fluorescent microscopy of human dendritic cells infected with VSV-expressing GFP. FIG. 12A is mock infected dendritic cells. FIG. 12B is 0.1 VSV-GFP (20X); FIG. 12C is 0.1 VSV-GFP (40X) and FIG. 12D is 0.1 VSV-GFP (40X).

### DETAILED DESCRIPTION OF THE INVENTION

VSV has been used as a vector to make HPV-virus like particles and HTLV-1 virus like particles and chimeric VSV viruses that could be used in immunodetection, vaccine and anticancer strategies related to HPV and HTLV-1 infection. Human papillomavirus (HPV) are DNA viruses that can cause warts and cervical cancer. Over 90% of cervical cancer is associated with HPV infection. VSV tumor therapy may useful in the treatment of HPV-associated disease. Recombinant VSV have been generated that express the structural proteins of HPV (L1, L2). Large amounts of these HPV proteins can now be made in VSV. In addition, the HPV structural proteins have recombined to form HPV-like particles (HPV without the genome or non-structural proteins that help HPV replication). Thus, these HPV-like particles look like real HPV (i.e., should be antigenically identical) though are replication-incompetent. These HPV-like particles are useful in vaccine strategies and/or immunodetection strategies to prevent or detect HPV infection, respectively. Other proteins of HPV, such as E2, E6 and E7 can be fused to these particles to broaden the potential response of the immune system to HPV.

VSV/HTLV-1 chimeric viruses have been made by inserting the HTLV-1 gag and env region (and other HTLV-1 proteins) into VSV. VSV containing the HTLV-1 proteins (chimeric VSV viruses) are used in vaccine protocols and as therapeutics. In addition, antigenically authentic HTLV-1 particles (gag/env) are made.

For example, these VSV/HPV chimeras are useful in ex-vivo HPV-related cancer vaccine and therapy strategies. VSV expressing HPV proteins or HPV-like particles can be used to infect/transduce antigen presenting cells such as dendritic cells to boost humoral and cell-mediated immune responses to HPV infection. This will result in vaccination to HPV or will generate HPV specific T-cell responses that could attack HPV-infected cells such as cervical cancer (i.e. cancer therapy). Without being bound by theory VSV/HPV will be presumed to preferentially replicate in cervical cancer cells and while replicating and destroying the malignant cells would express HPV proteins (structural and non-structural) that could stimulate anti-HPV cytotoxic T-cell (CTL) activity. The addition of immunomodulatory cassettes into VSV/HPV chimeras may even more enhance such CTL activity. The same strategy can be used against HTLV-1 mediated disease, using VSV/HTLV-1 technology.

Vesicular stomatitis virus (VSV) is a negative-stranded virus, comprising only 5 genes, that preferentially replicates in immortalized and malignant cells. Results of experiments disclosed herein demonstrate that a VSV vector comprising nucleic acid encoding human T cell leukemia virus type 1 (HTLV-1) gag and env proteins is capable of producing high levels of HTLV-1 virus like particles (VLPs) when cultured under suitable conditions. Results of experiments disclosed herein demonstrate that a VSV vector comprising nucleic acid encoding human papilloma virus (HPV) L1 capsid protein is capable of producing high levels of HPV virus like particles (VLPs) when cultured under suitable conditions. Data indicate that VSV-HPV-L1 infected mice contained evidence of cytotoxic T-cells specific to the L1 protein following vaccination. Results of experiments shown herein demonstrate that a VSV vector comprising nucleic acid encoding human T cell leukemia virus type I (HTLV-1) gag/pro and env proteins is capable of producing high levels of HTLV-1 virus like particles when cultured under suitable conditions.

Advantages of using a recombinant VSV system for production of VLPs for vaccine studies include that the VSV virus is relatively innocuous and naturally occurring human infections are rare. Accordingly, the apparent seroprevalence of VSV antibodies are generally low within the human population. Furthermore, VSV has a simple genetic constitution of only 5 genes (N, P, M, G, and L) and is unable to undergo reassortment or integration. The genetic malleability of VSV indicates that large, multiple inserts of foreign genes can be achieved that are expressed to high levels, without dramatically affecting virus growth. VSV has been found to elicit strong humoral and cellular immune responses and is able to elicit both mucosal and systemic immunity.

General Techniques

The practice of the present invention will employ, unless otherwise indicated, conventional techniques of molecular biology (including recombinant techniques), microbiology, cell biology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

For general information related to vesicular stomatitis virus, see, “Fundamental Virology”, second edition, 1991, ed. B. N. Fields, Raven Press, New York, pages 489-503; and “Fields Virology”, third edition, 1995, ed. B. N. Fields, vol. 1, pages 1121-1159.

“VSV” as used herein refers to any strain of VSV or mutant forms of VSV, such as those described in WO 01/19380. A VSV construct of this invention may be in any of several forms, including, but not limited to, genomic RNA, mRNA, cDNA, part or all of the VSV RNA encapsulated in the nucleocapsid core, VSV complexed with compounds such as PEG and VSV conjugated to a nonviral protein. VSV vectors of the invention encompasses replication-competent and replication-defective VSV vectors, such as, VSV vectors lacking G glycoprotein. Replication-defective VSV vectors can be grown in appropriate cell lines.

As used herein, a “virus like particle” or “VLP” refers to a viral capsid structure that comprises one or more of the structural proteins of a virus (DNA or RNA virus) responsible for virus capsid assembly that immunogenically and antigenically resembles conformation and shape of the virus. Virus like particles of the present invention do not contain replicative components and are not infectious. For example, a human papilloma virus like particle (referred to herein as “HPVLP” or “HPV VLP”) comprises the HPV structural protein L1 or L1 and L2. The present invention encompasses HPV structural proteins from any strain of HPV and in some examples the HPV strain is HPV strain 16, 18, 31 or 33 associated with cervical cancer. A HPV VLP may contain additional HPV viral proteins as long as the VLP is not infectious, that is, not capable of replication. A human T cell leukemia virus type 1 (HTLV-1) VLP may comprise part or all of a Gag (core protein) and a viral env protein and may comprises additional viral proteins, such as tax and rex, as long as the VLP is not infectious. In some examples, the viral env protein is HTLV-1 env protein. The present invention encompasses VLPs comprising HTLV-1 structural proteins from any strain of HTLV-1.

As used herein, the terms “malignant”, “malignant cells”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation. The term “tumors” includes metastatic as well as non-metastatic tumors.

As used herein “oncolytic activity” refers to inhibition or suppression of tumor and/or malignant and/or cancerous cell growth; regression of tumor and/or malignant and/or cancerous cell growth; cell death of tumor and/or malignant and/or cancerous cells or prevention of the occurrence of additional tumor and/or malignant and/or cancerous cells. As used herein, “inhibiting or suppressing tumor growth” refers to reducing the rate of growth of a tumor, halting tumor growth completely, causing a regression in the size of an existing tumor, eradicating an existing tumor and/or preventing the occurrence of additional tumors upon administration of the VSV comprising compositions, or methods of the present invention. “Suppressing” tumor growth indicates a growth state that is curtailed when compared to growth without contact with a VSV of the present invention. Tumor cell growth can be assessed by any means known in the art, including, but not limited to, measuring tumor size, determining whether tumor cells are proliferating using a ³H-thymidine incorporation assay, or counting tumor cells. “Suppressing” tumor and/or malignant and/or cancerous cell growth means any or all of the following states: slowing, delaying, and stopping tumor growth, as well as tumor shrinkage. “Delaying development” of tumor and/or malignant and/or cancerous cells means to defer, hinder, slow, retard, stabilize, and/or postpone development of the disease. This delay can be of varying lengths of time, depending on the history of the disease and/or individual being treated.

As used herein, the term “vector” refers to a polynucleotide construct designed for transduction/transfection of one or more cell types. VSV vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector. The present invention encompasses VSV vectors that comprise nucleic acid encoding viral structural proteins capable of assembling into VLPs.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P-NH2) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothioate linkage can be used in place of a phosphodiester linkage. Braun et al. (1988) J. Immunol. 141: 2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to a SEQ ID NO) also includes the complement sequence.

The following are non-limiting examples of polynucleotides: a gene or gene fragment, exons, introns, genomic RNA, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

“Under transcriptional control” is a term well understood in the art and indicates that transcription of a polynucleotide sequence depends on its being operably (operatively) linked to an element which contributes to the initiation of, or promotes, transcription. “Operably linked” refers to a juxtaposition wherein the elements are in an arrangement allowing them to function.

In the context of VSV, a “heterologous polynucleotide” or “heterologous gene” or “transgene” is any polynucleotide or gene that is not present in wild-type VSV.

In the context of VSV, a “heterologous” promoter is one which is not associated with or derived from VSV.

A “host cell” includes an individual cell or cell culture which can be or has been a recipient of a VSV vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected, transformed or infected in vivo or in vitro with a VSV vector of this invention.

“Replication” and “propagation” are used interchangeably and refer to the ability of an VSV vector of the invention to reproduce or proliferate. These terms are well understood in the art. For purposes of this invention, replication involves production of VSV proteins and is generally directed to reproduction of VSV. Replication can be measured using assays standard in the art. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

An “individual” is a vertebrate, preferably a mammal, more preferably a human. Mammals include, but are not limited to, farm animals, sport animals, rodents, primates, e.g. humans, and pets.

An “effective amount” is an amount sufficient to effect beneficial or desired results, including clinical results. An effective amount can be administered in one or more administrations. For purposes of this invention, an effective amount of a VSV vector is an amount that is sufficient to palliate, ameliorate, stabilize, reverse, slow or delay the progression of the disease state. In some examples, an “effective amount” of a VLP of the invention is an amount capable of eliciting an immune response when administered to an individual.

“Expression” includes transcription and/or translation.

As used herein, the term “comprising” and its cognates are used in their inclusive sense; that is, equivalent to the term “including” and its corresponding cognates.

“A,” “an” and “the” include plural references unless the context clearly dictates otherwise.

VSV

VSV sequences and constructs

VSV, a member of the Rhabdoviridae family, is a negative-stranded virus that replicates in the cytoplasm of infected cells, does not undergo genetic recombination or reassortment, has no known transforming potential and does not integrate any part of it genome into the host. VSV comprises an about 11 kilobase genome that encodes for five proteins referred to as the nucleocapsid (N), polymerase proteins (L) and (P), surface glycoprotein (G) and a peripheral matrix protein (M). The genome is tightly encased in nucleocapsid (N) protein and also comprises the polymerase proteins (L) and (P). Following infection of the cell, the polymerase proteins initiate the transcription of five subgenomic viral mRNAs, from the negative-sense genome, that encode the viral proteins. The polymerase proteins are also responsible for the replication of the full-length viral genomes that are packaged into progeny virions. The matrix (M) protein binds to the RNA genome/nucleocapsid core (RNP) and also to the glycosylated (G) protein, which extends from the outer surface in an array of spike like projections and is responsible for binding to cell surface receptors and initiating the infectious process.

Following attachment of VSV through the (G) protein to receptor(s) on the host surface, the virus penetrates the host and uncoats to release the RNP particles. The polymerase proteins, which are carried in with the virus, bind to the 3′ end of the genome and sequentially synthesize the individual mRNAs encoding N, P, M, G, and L, followed by negative-sense progeny genomes. Newly synthesized N, P and L proteins associate in the cytoplasm and form RNP cores which bind to regions of the plasma membrane rich in both M and G proteins. Viral particles form and budding or release of progeny virus ensues.

A table of various VSV strains is shown in “Fundamental Virology”, second edition, supra, at page 490. WO 01/19380 and U.S. Pat. No. 6,168,943 disclose that strains of VSV include Indiana, New Jersey, Piry, Colorado, Coccal, Chandipura and San Juan. The complete nucleotide and deduced protein sequence of a VSV genome is known and is available as Genbank VSVCG, accession number J02428; NCBI Seq ID 335873; and is published in Rose and Schubert, 1987, in The Viruses: The Rhabdoviruses, Plenum Press, NY. pp. 129-166. A complete sequence of a VSV strain is shown in U.S. Pat. No. 6,168,943. VSV New Jersey strain is available from the American Type Culture Collection (ATCC) and has ATCC accession number VR-159. VSV Indiana strain is available from the ATCC and has ATCC accession number VR-1421.

The present invention encompasses the use of any strain of VSV, including mutants of VSV disclosed in WO 01/19380. The present invention encompasses any form of VSV, including, but not limited to genomic RNA, mRNA, cDNA, and part or all of VSV RNA encapsulated in the nucleocapsid core. The present invention encompasses VSV in the form of a VSV vector construct as well as VSV in the form of viral particles. The present invention also encompasses nucleic acid encoding specific VSV vectors disclosed herein. As discussed herein, VSV vectors of the present invention encompass replication-competent as well as replication-defective VSV vectors.

The present invention encompasses VSV vectors comprising nucleic acid encoding part or all of HTLV-1 gag/pro and HTLV-1 env proteins. The present invention also encompasses VSV vectors comprising nucleic acid encoding HPV-L1 or L1 and L2 viral proteins. The present invention encompasses any strain of HTLV-1 or HPV. In some examples, the HPV strain is HPV strain 16, 18, 31, 33 or 45 associated with cervical cancer.

In some examples, the VSV vector lacks a protein function essential for replication, such as G-protein function or M and/or N protein function. The VSV vector may lack several protein functions essential for replication. The present invention also provides viral particles comprising a VSV vector of the present invention. The present invention also comprises isolated nucleic acid encoding a recombinant VSV vector of the present invention as well as host cells comprising a recombinant VSV vector of the present invention.

VSV replicates preferentially in malignant cells. This is primarily due to host defense mechanisms that normally contain VSV infection being damaged in cancerous cells, thus allowing the virus to propagate. The virus will destroy the malignant cells by mechanisms involving virus-induced apoptosis. Table 2 provides a list of cell lines and VSV ability to replicate in these cell lines.

TABLE 2

| Cell line | Cell or tissue type | VSU infection |
|---|---|---|
| BHK | hamster kidney | + |
| HMVEC | human normal | − |
| B16 (F10) | Melanoma | + |
| DA-3 | Breast | + |
| MCF-7 | transformed hu breast | + |
| BC-1 | hu hematological malignancy | + |
| Jurkat | hu hematological malignancy | + |
| HL60 | hu hematological malignancy | + |
| K562 | hu hematological malignancy | + |
| PC-3 | hu transformed prostate | + |
| Hela | hu cervical tumor | + |

wherein “hu” refers to of human origin

Recombinant VSVs efficiently produce large amounts of difficult to make/toxic/rare proteins. As disclosed in the present patent application recombinant VSV produce large amounts of HPV VLP. Thus, such viruses could be useful in making large amounts of VLPs and other toxic or hard to make proteins. Accordingly, the invention provides VSV vectors comprising nucleic acid encoding part or all of HTLV-1 gag protein, or part or all of HTLV-1 gag protein and pro protein and viral env proteins, such as for example, HTLV-1 env protein, for use in producing VSV vectors or VSV viral particles or VSV produced VLPs. The present invention also encompasses VSV vectors comprising nucleic acid encoding HPV-L1 or L1 and L2 viral proteins for use in producing VSV vectors or VSV viral particles or VSV produced VLPs. The present invention encompasses any strain of HTLV-1 or HPV. In some examples, the HPV strain is HPV strain 16, 18, 31, 33 or 45 associated with cervical cancer. VSV vectors and VSV viral particles can be generated to make VLPs, in large amounts and constitutes a eukaryotic version of the baculovirus/insect cell expression system. Advantages of the VSV system for production of VLP include high level of expression and authentic (eukaryotic) processing, unlike in insect cells.

Accordingly, the present invention provides methods for making a recombinant VSV vector of the present invention comprising growing a cell comprising said VSV vector under conditions whereby VSV is produced; and optionally isolating said VSV. In some examples, the VSV vector is replication defective and the host cells comprising the VSV protein function essential for VSV replication such that said VSV vector is capable of replication in said host cell. In some examples, the VSV vector comprises nucleic acid encoding a viral capsid protein, such as HPV-L1. In other examples, the VSV vector comprises nucleic acid encoding part or all of a HTLV-1 gag protein and/or part or all of a viral env protein, such as HTLV-1 env protein. Accordingly, the present invention provides methods for producing HTLV-1 VLPs comprising growing a cell comprising a VSV vector comprising nucleic acid encoding part or all of a HTLV-1 gag or gag/pro protein and/or a viral env protein, such as HTLV-1 env protein, wherein said part or all of HTLV-1 gag or gag/pro protein and said part or all of viral env protein are capable of assembling into a HTLV-1 VLP. In some embodiments of the method, the VSV vector comprises additional HTLV-1 viral proteins or immunomodulatory proteins, such as cytokines. Accordingly, the present invention provides methods for producing HPV VLPs comprising growing a cell comprising a VSV vector comprising nucleic acid encoding a HPV L1 or L1 and L2 protein, wherein said HPV L1 or L1 and L2 protein are capable of assembling into a HPV VLP. In some embodiments of the method, the VSV vector comprises additional HPV viral proteins or immunomodulatory proteins, such as cytokines.

HTLV-1

Human T Cell Leukemia Virus Type I (HTLV-1) is the etiologic agent of adult T cell leukemia/lymphoma (ATL) and tropical spastic parapesis. HTLV-1, a positive stranded RNA virus of the Oncovirinae family infects between 10 and 20 million people worldwide. The virus causes at least 2 types of disease: a highly aggressive T cell malignancy, adult T cell leukemia/lymphoma (ATL) and a variety of chronic inflammatory syndromes, most notably HTLV-1 associated myelopathy also known as tropical spastic parapesis (TSP/HAM). The major modes of virus transmission are through sexual intercourse, infected blood products or from mother to child in breast milk.

The genome of HTLV-1, a type C retrovirus, encodes two structural proteins Gag and Env, a polymerase protein and two regulatory proteins tax and rex. A schematic depiction of the structure and organization of the HTLV genome is given in Fields Virology, Third Edition, vol. 2, Ed. Fields et al., pub. Lippincott-Raven, page 1850. The HTLV-1 envelope glycoprotein which plays a crucial role in the infectious process, is synthesized as a precursor (gp62) that is cleaved into two glycoproteins (gp46 and gp21). The gp46 subunit is expressed on the surface of viral particles and is involved in the specific attachment to an undefined cellular receptor on CD4 T cells. The gp21 subunit allows anchorage of the envelope to viral or cellular membranes and facilitates cell fusion. The HTLV-1 Gag-Pro genes are expressed as two polyprotein precursors. The viral Protease cleaves the Gag precursor into the matrix protein (p19), a capsid protein (p24) and a nucleocapsid protein (p15). Studies with other retroviruses indicates that expression of the Gag-Pro genes leads to secretion of mature virus like particles (I. Le Blanc et al. (2001, Virus Res 78:5-16); R. H. Takahashi et al. (1999, Virology 256:371-80). A complete human T-lymphotropic virus 1 genome is provided in NCBI having Accession # NC_{—}001436.

Both cell mediated and humoral immunity are thought to be important in controlling infection. In infected individuals, antibodies against the Gag protein are the first to appear after infection followed by anti-Env antibodies. While the gag proteins are the major immunogens, antibodies are also directed against both gp46 and to some extent gp21. The CTL response to HTLV-1 is also crucial in limiting viral replication. Anti-HTLV-1 CTLs are extremely abundant and most recognize a single viral protein, tax (Fields Virology (1996, Raven, Philadelphia, p. 1849-1869).

The present invention encompasses replication-defective, replication-competent, and mutant forms of VSV expressing one or more HTLV-1 viral structural proteins, such as part or all of gag and/or part or all of env, wherein the HTLV-1 can be any strain, and may additionally express other HTLV-1 viral proteins, such as for example, part or all of HTLV-1 tax or rex proteins. The present invention encompasses VSV that expresses all or parts of HTLV-1 tax and/or rex with or without the HTLV-1 viral structural proteins such as env and/or gag for use in eliciting an immune response. Without being bound by theory, such a virus would induce cell-mediated and humoral activity to the structural and/or non-structural proteins to give a broader, multivalent immune response. For example, nucleic acid encoding HTLV-1 gag may be fused to all or parts of nucleic acid encoding tax and/or rex, such as for example, to produce gag-tax/env VLPs. In some examples, the VSV comprises nucleic acid encoding HTLV-1 gag and pro and HTLV-1 env proteins. Upon expression, the HTLV-1 pro cleaves the HTLV-1 gag into matrix protein (p19), a capsid protein (p24) and a nucleocapsid protein (p15). In other examples, the VSV vector comprises part(s) of gag, such as p19, p24, or p15, as long as the parts are capable of forming a VLP. In other examples, the VSV expresses HTLV-1 gag and pro and non-HTLV-1 viral env protein as long as the non-HTLV-1 viral env protein is capable of forming a VLP along with HTLV-1 gag. In some examples, a VSV vector is constructed wherein nucleic acid encoding VSV G protein is deleted and replaced with nucleic acid encoding HTLV-1 env. Such a construct would be produced by fusing parts of the VSV G transmembrane region onto the HTLV-1 env region such that when VSV buds from the cell, the HTLV-1 env will be inserted into the VSV particle. In other examples, VSV chimeric viruses are made that express both HTLV-1 env and VSV G protein. In yet other examples, HTLV-1 VLPs are generated that comprise VSV G on their surface, such as by fusing the gag/env interacting transmembrane region of HTLV-1 env to VSV G. VSV G protein is tropic for a number of tissue types and may be additionally immunogenic. In other examples, HTLV-1 VLPS could be produced that have heat shock proteins such as gp70 or gp96 on their surface. Heat shock proteins fused to transmembrane regions of HTLV-1 env will be associated on the surface of VLPs. Without being bound to theory, such a VLP may increase immunogenicity and target antigen presenting cells such as dendritic cells. In other examples, a VSV construct comprising a HTLV-1 nucleic acid encoding a structural protein may also comprise nucleic acid encoding an immunomodulatory protein, such as for example, an interferon, such as interferon-beta; an interleukin, such as interleukin 2 or 12, or a chemokine or chemoattractant. Accordingly, the present invention provides VSV produced HTLV-1 VLPs comprising HTLV-1 gag and env proteins for use in eliciting an immune response in individuals, such as in vaccine protocols.

HPV-1

Papillomaviruses are a family of small DNA viruses encoding up to eight early (E1, E2, E3, E4, E5, E6, E7 and E8) and two late genes (L1 and L2). Human papillomavirus (HPV) is a major etiologic agent of genital warts and cervical cancer. A clinicopathological grouping of HPV and the malignant potential of the lesions with which they are most frequently associated are summarized in “Papillomaviruses and Human Cancer” by H. Pfister, CRC Press, Inc. (1990). For example, HPV type 1 (HPV-1) is present in plantar warts. HPV-16 and HPV-18 are associated with cervical carcinomas (Reuter et al., 2002, Journal of Virology, vol. 76:p 8900-8909 and U.S. Pat. No. 6,365,160). HPV-33 was cloned from an invasive cervical carcinoma using HPV-16 as a probe under conditions of reduced stringency. US patent 6,344,314 discloses the DNA sequence of HPV-33 and describe its relationship to HPV-16.

Human papillomavirus (HPV), a nonenveloped, double-stranded DNA virus of the papovavirus family, is a major etiologic agent of cervical cancer. HPV comprises a single molecule of circular dsDNA of approximately 8 kb contained within a spherical protein coat, or capsid, and encodes about 10 viral products (Fields Virology (1996, Third Edit Lippincott-Raven Publishers, Philadelphia, p. 2045-2109). The capsid is composed of 72 capsomeres consisting of two structural proteins, namely L1 of an approximate molecular weight of 64 kDa (HPV-18), which represents approximately 80% of the total coat protein, and a 70 kDa protein referred to as L2 (R. Kirnbauer et al. (1993, J Virol 67:6929-36). More than 70 HPV types are now recognized, many of which, such as HPV 6 and 11, cause genital warts and infect other mucosal sites such as the respiratory tract, oral cavity and the conjunctiva. Other strains of HPV, such as 16, 18, 45 and 31 are strongly associated (>95%) with cervical cancer (Fields Virology (1996, Third Edit Lippincott-Raven Publishers, Philadelphia, p. 2077-2109). Three independent transforming proteins referred to as early genes E5, E6 and E7 are encoded by papillomaviruses. In most HPV-positive cancers however, E5 whose functions remains unclear is not significantly expressed. In contrast, both the HPV-encoded proteins E6, and E7, which inhibit the tumor suppressor p53 and Rb respectively, are required for the efficient immortalization of primary human fibroblasts (H. L. Greenstone et al. (1998, Proc Natl Acad Sci USA 95:1800-5); Fields Virology (1996, Third Edit Lippincott-Raven Publishers, Philadelphia, p. 2077-2109).

Cervical cancer constitutes approximately 7% of all cancers in women in industrialized nations (24% in developing countries). The recognition that HPV infections are major etiological agents of cervical cancer has led to efforts to prevent the cancer by immunization against HPV (M. R. Hilleman (2000, J Clin Virol 19:79-90). Based on the pathogenesis of HPV infection and disease, two main strategies have been proposed for the development of a successful HPV vaccine. The first strategy is to prime neutralizing antibodies, preferentially at the mucosal (and cutaneous) sites, so that infection of epithelial cells can be prevented (N. D. Christensen et el. (2001, Virology, 291:324-34); V. Revaz et al. (2001, Virology 279:354-60). A second strategy is to elicit HPV-specific T cells, as virus-specific T cells have been shown to be important for effectively controlling and eradicating numerous viral infections (T. Giroglou et al. (2001, Vaccine 19:1783-93); J. C. Steele et al. (2002, J Virol 76:6027-36); O. M. Williams et al. (2002, J Virol 76:7418-29). Accordingly, the present invention provides VSV produced HPV VLPs comprising HPV L1 or HPV L1 and L2 protein for use in elicting an immune response in individuals, such as in vaccine protocols.

When expressed in bacteria, yeast or eukaryotic cells, the papillomavirus capsid protein L1, alone or in combination with L2 autoassembles to form intact virus-like particles (VLPs) that morphologically and antigenically resemble native virion (Chen et al., 2001, J. Mol. Biol. Vol. 307:173-182). immunization of animals with various papilloma based VLP based vaccines has been shown to elicit high antibody titre and durable T-cell responses. The presence of vaccine-induced neutralizing antibodies was shown to correlate with complete protection against viral challenge in the cottontail rabbit papillomavirus model, the canine oral papilloma virus dog model and the bovine papillomavirus (BPV) cow model (N. D. Christensen et el. (2001, Virology, 291:324-34); R. Kimbauer et al. (1992, Proc Natl Acad Sci U S A 89:12180-4); J. D. Reuter et al. (2001, J Virol Methods 98:127-34); V. Revaz et al. (2001, Virology 279:354-60).

The present invention encompasses replication-defective, replication-competent, and mutant forms of VSV expressing one or more HPV viral structural proteins, such as L1 or L1 and L2, wherein the HPV can be any strain, and may additionally express other HPV viral proteins, such as for example, HPV E2, E6 or E7. The present invention encompasses VSV that expresses all or parts of HPV E2 and/or E6 and/or E7 with or without the HPV viral structural proteins such as L1 and/or L2 for use in eliciting an immune response. For example, nucleic acid encoding HPV L1 protein may be fused to all or parts of nucleic acid encoding E2 and/or E6 and/or E7, such as for example, to produce HPV L1 E2, or E6 or E7 VLPs. In some examples, the VSV comprises nucleic acid encoding HPV L1 from a strain of HPV associated with cervical cancer such as for example, HPV 16. HPV 16 DNA sequence is disclosed in Seedorfet al. (1985, Virology, 145(1), 181-185). In other examples, the VSV expresses chimeric HPV VLPs such as for example, VSV expressing L1 from HPV 16 and 18, or along with combinations of other HPV proteins, such as E2 and/or E6 and/or E7, such that immune responses to different strains could be generated from one or more recombinant VSV (rVSV). For example, the L1 from HPV 16 and HPV 18 could be inserted into one VSV construct, or into separate constructs. In some examples, a VSV vector is constructed wherein nucleic acid encoding HPV L1 is fused to VSV G protein. VSV G protein is tropic for a number of tissue types and may be additionally immunogenic. In other examples, HPV VLPS could be produced that have heat shock proteins such as gp70 or gp96 on their surface. In other examples, a VSV construct comprising a HPV nucleic acid encoding a structural protein may also comprise nucleic acid encoding an immunomodulatory protein, such as for example, an interferon, such as interferon-beta; an interleukin, such as interleukin 2 or 12, or a chemokine or chemoattractant.

Host cells, compositions and kits comprising VSV

The present invention also provides host cells comprising (i.e., transformed, transfected or infected with) the VSV vectors or virus particles or VLPs described herein. Both prokaryotic and eukaryotic host cells, including insect cells, can be used as long as sequences requisite for maintenance in that host, such as appropriate replication origin(s), are present. For convenience, selectable markers are also provided. Host systems are known in the art and need not be described in detail herein. Prokaryotic host cells include bacterial cells, for example, E. coli, B. subtilis, and mycobacteria. Among eukaryotic host cells are yeast, insect, avian, plant, C. elegans (or nematode) and mammalian host cells. Examples of fungi (including yeast) host cells are S. cerevisiae, Kluyveromyces lactis (K. lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, mouse L cells, LNCaP cells, Chinese hamster ovary (CHO) cells, human embryonic kidney (HEK) cells, and African green monkey cells. Xenopus laevis oocytes, or other cells of amphibian origin, may also be used.

The present invention also includes compositions, including pharmaceutical compositions, containing the VSV vectors described herein, or the VSV produced VLPs, such as HPV-VLPs and HTLV-1 VLPs described herein. Such compositions are useful for administration in vivo, for example, for eliciting an immune response in an individual. Compositions can comprise a VSV vector described herein or a VSV produced VLP and a suitable solvent, such as a physiologically acceptable buffer. These are well known in the art. In other embodiments, these compositions further comprise a pharmaceutically acceptable excipient. These compositions, which can comprise an effective amount of a VSV produced VLP in a pharmaceutically acceptable excipient, are suitable for systemic or local administration to individuals in unit dosage forms, sterile parenteral solutions or suspensions, sterile non-parenteral solutions or oral solutions or suspensions, oil in water or water in oil emulsions and the like. Formulations for parenteral and nonparenteral drug delivery are known in the art and are set forth in Remington's Pharmaceutical Sciences, 19th Edition, Mack Publishing (1995). Compositions also include lyophilized and/or reconstituted forms of the VSV vectors (including those packaged as a virus) of the invention.

The present invention also encompasses kits containing VSV vector(s) of this invention. These kits can be used for example for producing proteins for screening, assays and biological uses, such as treating cancer. Procedures using these kits can be performed by clinical laboratories, experimental laboratories, medical practitioners, or private individuals.

The kits of the invention comprise a VSV vector described herein in suitable packaging. The kit may optionally provide additional components that are useful in the procedure, including, but not limited to, buffers, developing reagents, labels, reacting surfaces, means for detection, control samples, instructions, and interpretive information. The kit may include instructions for administration of a VSV vector.

Methods of producing recombinant VSV

The study of VSV and related negative strand viruses has been limited by the inability to perform direct genetic manipulation of the virus using recombinant DNA technology. The difficulty in generating VSV from DNA is that neither the full-length genomic nor antigenomic RNAs are infectious. The minimal infectious unit is the genomic RNA tightly bound to 1,250 subunits of the nucleocapsid (N) protein (Thomas et al., 1985, J. Virol. 54:598-607) and smaller amounts of the two virally encoded polymerase subunits, L and P. To reconstitute infectious virus from the viral RNA, it is necessary first to assemble the N protein-RNA complex that serves as the template for transcription and replication by the VSV polymerase. Although smaller negative-strand RNA segments of the influenza virus genome can be packaged into nucleocapsids in vitro, and then rescued in influenza infected cells (Enami et al., 1990, Proc. Natl. Acad. Sci. USA 87:3802-3805; Luytjes et al., 1989, Cell 59:1107-1113), systems for packaging the much larger eukaryotic genomic RNAs in vitro are not yet available.

Systems for replication and transcription of DNA-derived minigenomes or small defective RNAs from Rhabdoviruses (Conzelmann and Schnell, 1994, J. Virol. 68:713-719; Pattnaik et al., 1992, Cell 69:1011-1120) have been described. In these systems, RNAs are assembled into nucleocapsids within cells that express the viral N protein and polymerase proteins. These systems do not allow genetic manipulation of the full-length genome of infectious viruses. U.S. Pat. No. 6,168,943 discloses methods for the preparation of infectious recombinant vesiculovirus capable of replication in an animal into which the recombinant vesiculovirus is introduced. For example, U.S. Pat. No. 6,168,943 describes that vesiculoviruses are produced by providing in an appropriate host cell: (a) DNA that can be transcribed to yield (encode) vesiculovirus antigenomic (+) RNA (complementary to the vesiculovirus genome), (b) a recombinant source of vesiculovirus N protein, (c) a recombinant source of vesiculovirus P protein, and (d) a recombinant source of vesiculovirus L protein; under conditions such that the DNA is transcribed to produce the antigenomic RNA, and a vesiculovirus is produced that contains genomic RNA complementary to the antigenomic RNA produced from the DNA.

Alternatively, after purification of genomic RNA, VSV mRNA can be synthesized in vitro, and cDNA prepared by standard methods, followed by insertion into cloning vectors (see, e.g., Rose and Gallione, 1981, J. Virol. 39(2):519-528). VSV or portions of VSV can be prepared using oligonucleotide synthesis (if the sequence is known) or recombinant methods (such as PCR and/or restriction enzymes). Polynucleotides used for making VSV vectors of this invention may be obtained using standard methods in the art, such as chemical synthesis, recombinant methods and/or obtained from biological sources. Individual cDNA clones of VSV RNA can be joined by use of small DNA fragments covering the gene junctions, generated by use of reverse transcription and polymerase chain reaction (RT-PCR) (Mullis and Faloona, 1987, Meth. Enzymol. 155:335-350) from VSV genomic RNA (see Section 6, infra). The ability to recover fully infectious virus from a plasmid cDNA copy of the VSV genome has allowed genetic manipulation of this virus to become feasible.

In an example disclosed herein, a cDNA clone representing the entire 11,161 nucleotides of VSV has been generated and unique Xho I/Nhe I sites were added to facilitate entry of a heterologous gene, e.g. for example, HPV-L1. Transcription of the cDNA is dependent on T7 RNA polymerase. Vaccinia vTF7-3 was used to infect baby hamster kidney cells (BHK-21), to provide a source of polymerase. Subsequently, VSV cDNA was transfected into the same cells together with three other plasmids that express the VSV N, P and L proteins. These latter three proteins facilitate the assembly of nascent VSV antigenomic RNA into nucleocapsids and initiate the VSV infectious cycle. After 24 hours, host cells were lysed, clarified and residual vaccinia removed by filtration through a 0.2 um filter onto fresh BHK cells. Only recombinant VSVs are produced by this method since no wild-type VSV can be generated (Rose et al., 1995, P.N.A.S. USA).

VSV may be genetically modified in order to alter it properties for use in vivo. Methods for the genetic modification of VSV are well established within the art. For example, a reverse genetic system has been established for VSV (Roberts et al., Virology, 1998, 247:1-6) allowing for modifications of the genetic properties of the VSV. Standard techniques well known to one of skill in the art may be used to genetically modify VSV and introduce desired genes within the VSV genome to produce recombinant VSVs (see for example, Sambrooke et al., 1989, A Laboratory Manual, New York: Cold Spring Harbor Laboratory Press. For insertion of nucleotide sequences into VSV vectors, for example nucleotide sequences encoding a HTLV-1 or HPV viral protein, or for VSV gene sequences inserted into vectors, such as for the production helper cell lines, specific initiation signals are required for efficient translation of inserted protein coding sequences. These signals include the ATG initiation codon and adjacent sequences. In cases where an entire VSV gene, such as G-protein including its own initiation codon and adjacent sequences are inserted into the appropriate vectors, no additional translational control signals may be needed. However, in cases where only a portion of the gene sequence is inserted, exogenous translational control signals, including the ATG initiation codon, must be provided. The initiation codon must furthermore be in phase with the reading frame of the protein coding sequences to ensure translation of the entire insert. These exogenous translational control signals and initiation codons can be of a variety of origins, both natural and synthetic. Nucleic acid encoding HTLV-1 gag/pro and env or HPV L1 or L1 and L2 can be obtained by recombinant methods, from naturally occurring forms or by chemical synthesis.

Following infection of a host cell, recombinant VSV shuts down host cell protein synthesis and expresses not only its own five gene products, but also heterologous proteins encoded within its genome. Successful expression of heterologous nucleic acid from VSV recombinants requires only the addition of the heterologous nucleic acid sequence into the full-length cDNA along with the minimal conserved sequence found at each VSV gene junction. This sequence consists of the polyadenylation/transcription stop signal (3′ AUACU₇) followed by an intergenic dinucleotide (GA or CA) and a transcription start sequence (3′ UUGUCNNUAG) complementary to the 5′ ends of all VSV mRNAs. Ball et al. 1999, J. Virol. 73:4705-4712; Lawson et al. 1995, P.N.A.S. USA 92:4477-4481; Whelan et al. 1995, P.N.A.S. USA 92:8388-8392. Additionally, restriction sites, preferably unique, (e.g., in a polylinker) are introduced into the VSV cDNA, for example in intergenic regions, to facilitate insertion of heterologous nucleic acid, such as nucleic acid encoding an interleukin or interferon. In other examples, the VSV cDNA is constructed so as to have a promoter operatively linked thereto. The promoter should be capable of initiating transcription of the cDNA in an animal or insect cell in which it is desired to produce the recombinant VSV vector. Promoters which may be used include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); heat shock promoters (e.g., hsp70 for use in Drosophila S2 cells); the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); and myosin light chain-2 gene control region which is active in skeletal muscle (Sani, 1985, Nature 314:283-286). Preferably, the promoter is an RNA polymerase promoter, preferably a bacteriophage or viral or insect RNA polymerase promoter, including but not limited to the promoters for T7 RNA polymerase, SP6 RNA polymerase, and T3 RNA polymerase. If an RNA polymerase promoter is used in which the RNA polymerase is not endogenously produced by the host cell in which it is desired to produce the recombinant VSV, a recombinant source of the RNA polymerase must also be provided in the host cell. Such RNA polymerase are known in the art.

The VSV cDNA can be operably linked to a promoter before or after insertion of nucleic acid encoding a heterologous protein, such as a HTLV-1 or HPV viral protein. In some examples, a transcriptional terminator is situated downstream of the VSV cDNA. In other examples, a DNA sequence that can be transcribed to produce a ribozyme sequence is situated at the immediate 3′ end of the VSV cDNA, prior to the transcriptional termination signal, so that upon transcription a self-cleaving ribozyme sequence is produced at the 3′ end of the antigenomic RNA, which ribozyme sequence will autolytically cleave (after a U) this fusion transcript to release the exact 3′ end of the VSV antigenomic (+) RNA. Any ribozyme sequence known in the art may be used, as long as the correct sequence is recognized and cleaved. (It is noted that hammerhead ribozyme is probably not suitable for use.)

VSV vectors of the present invention comprise one or more heterologous nucleic acid sequence(s) encoding a viral structural protein. In examples disclosed herein, a VSV vector comprises heterologous nucleic acid sequences encoding a HPV capsid protein, such as L1 or L1 and L2. In other examples, a VSV vector comprises heterologous nucleic acid sequences encoding part or all of a HTLV-1 gag-pro and part or all of env protein.

The present invention encompasses expression systems comprising a VSV vector comprising one or more heterologous nucleotide sequence(s), such as, a nucleotide sequence encoding a HPV L1 or L1 and L2, or a nucleotide sequence encoding a HTLV-1 gag and env protein inserted within a region of the VSV essential for replication, such as the G glycoprotein region, or other region essential for replication, such that the VSV lacks the essential function and is replication-defective. The VSV vector may have a mutation, such as a point mutation or deletion of part or all, of any region of the VSV genome, including the G, M, N, L or P region. If the mutation is in a region essential for replication, the VSV will be grown in a helper cell line that provides the essential region function. The VSV may also comprise a mutation, such as for example, a point mutation or deletion of part or all of a nucleotide sequence essential for replication, and optionally, with the heterologous nucleotide sequence inserted in the site of the deleted nucleotide sequence. The heterologous nucleotide sequence may be operably linked to a transcriptional regulatory sequence. Following replication-defective VSV infection of a cell, progeny viruses will lack essential protein function and cannot disseminate to infect surrounding tissue. In additional embodiments, the VSV vector is mutated in nucleic acid, such as by point mutation, substitution or addition of nucleic acid, or deletion of part or all, of nucleic acid encoding other VSV protein function such as, M protein and/or N protein function. VSV may be targeted to a desired site in vitro to increase viral efficiency. For example, modification of VSV G protein (or other VSV proteins) to produce fusion proteins that target specific sites may be used to enhance VSV efficiency in vivo.. Such fusion proteins may comprise, for example, but not limited to single chain Fv fragments that have specificity for tumor antigens. (Lorimer et al., P.N.A.S. U.S.A., 1996. 93:14815-20).

A VSV vector lacking a gene(s) essential for viral replication can be grown in an appropriate complementary cell line. Accordingly, the present invention provides recombinant helper cell lines or helper cells that provide a VSV protein function essential for replication of a replication-deficient VSV construct. In some examples, the protein function is G-protein function. For example, a VSV vector comprising nucleic acid encoding a HTLV-1 gag and env protein and lacking G-protein function can be grown in a cell line, i.e., a helper cell line, for example, a mammalian cells line such as CHO cell line, permissive for VSV replication, wherein said cell line expresses an appropriate G-protein function, such that said VSV is capable of replicating in the cell line. These complementing or helper cell lines are capable of allowing a replication-defective VSV to replicate and express one or more foreign genes or fragments thereof encoded by the heterologous nucleotide sequence. In some embodiments, the VSV vector lacks a protein function essential for replication, such as for example, G-protein function and the host cell line comprises nucleic acid encoding the protein function essential for replication, such as for example, VSV G-protein function. Complementing cell lines can provide VSV viral function through, for example, co-infection with a helper virus, or by integration or otherwise maintaining in stable form part or all of a viral genome encoding a particular viral function. In other examples, additional VSV non-essential proteins can be deleted or heterologous nucleotide sequences inserted into nucleotide regions encoding non-essential VSV, such as for example, the M and N proteins. The heterologous nucleotide sequence can be inserted into a region non-essential for replication wherein the VSV is replication-competent. Heterologous nucleotide sequences can be inserted in non-essential regions of the VSV genome, without necessitating the use of a helper cell line for growth of the VSV vector.

The recombinant VSV of the invention are produced for example, by providing in an appropriate host cell VSV cDNA wherein said cDNA comprises nucleotide sequence encoding a heterologous protein, such as for example, a HPV L1 or L1 and L2 or HTLV-1 gag protein. The nucleic acid encoding a heterologous protein can be inserted in a region non-essential for replication, or a region essential for replication, in which case the VSV is grown in the presence of an appropriate helper cell line. In some examples, the production of recombinant VSV vector is in vitro, in cell culture, in cells permissive for growth of the VSV. Standard recombinant techniques can be used to construct expression vectors containing DNA encoding VSV proteins. Expression of such proteins may be controlled by any promoter/enhancer element known in the art. Promoters which may be used to control expression of VSV proteins can be constitutive or inducible.

The host cell used for recombinant VSV production can be any cell in which VSV grows, e.g., mammalian cells and some insect (e.g., Drosophila) cells. Primary cells lacking a functional INF system, or in other examples, immortilized or tumor cell lines can be used. A vast number of cell lines commonly known in the art are available for use. By way of example, such cell lines include but are not limited to BHK (baby hamster kidney) cells, CHO (Chinese hamster ovary) cells, HeLA (human) cells, mouse L cells, Vero (monkey) cells, ESK-4, PK-15, EMSK cells, MDCK (Madin-Darby canine kidney) cells, MDBK (Madin-Darby bovine kidney) cells, 293 (human) cells, and Hep-2 cells. Such cell lines are publicly available for example, from the ATCC and other culture depositories.

Recombinant VSV (rVSV) produced by cell lines can be isolated using for example, an affinity matrix. Method of isolating VSV by affinity matrix are described in for example, WO 01/19380. Briefly, methods for isolating a rVSV comprises adding the VSV to an affinity matrix, to produce bound VSV, washing the bound VSV, and eluting the VSV from the affinity matrix. The present invention encompasses a modified VSV that comprises a non-naturally occurring fusion protein on the outer surface of the virus. The non-native protein may be a fusion protein comprising an affinity tag and a viral envelope protein or it may be derived from a producer cell. Producer cell lines may be engineered to express one or more affinity tags on their plasma membranes which would be acquired by the virus as it buds through the membrane. One example of an affinity tag is the use of Histidine residues which bind to immobilized nickel columns. Affinity tags also include antibodies. Other protocols for affinity purification may be used as known within the art, for example, but not limited to, batch processing, a solution of virus and affinity matrix, pelleting the VSV-bound matrix by centrifugation, and isolating the virus. Alternatively, VSV can be collected and purified as described in U.S. Pat. No. 6,168,943. Briefly, VSV is collected from culture supernatants, and the supernatants clarified to remove cellular debris. One method of isolating and concentrating the virus is by passage of the supernatant through a tangential flow membrane concentration. The harvest can be further reduced in volume by pelleting through a glycerol cushion and by concentration on a cesium chloride or sucrose step gradient or other form of gradient.

The present invention also provides methods of producing a HTLV-1 virus like particle (VLP) comprising growing a cell comprising a VSV vector comprising isolated nucleic acid encoding part or all of a HTLV-1 Gag gene and/or part or all of a viral Env gene under conditions suitable for expression of the HTLV-1 Gag and viral Env and -assembly into a VLP, and optionally isolating said VLP. The VSV vector may further comprise nucleic acid encoding a HTLV-1 pol protein. The present invention also provides methods of producing a HTLV-1 VLP comprising growing a cell expressing viral env function, wherein the nucleic acid encoding the function is integrated in the cells genome or exists extrachromasomally, and comprising a VSV vector comprising part or all of a HTLV-1 Gag gene under conditions suitable for expression of the HTLV-1 Gag and viral Env and assembly into a VLP, and optionally isolating said VLP. The present invention also provides methods of producing a HTLV-1 VLP comprising growing a cell expressing HTLV-1 gag function, wherein the nucleic acid encoding the function is integrated in the cells genome or exists extrachromasomally, and comprising a VSV vector comprising part or all of a viral env gene under conditions suitable for expression of the HTLV-1 Gag and viral Env and assembly into a VLP, and optionally isolating said VLP. The present invention also provides methods of producing a HPV virus like particle (VLP) comprising growing a cell comprising a VSV vector comprising nucleic acid encoding a HPV L1 or L1 and L2 protein under conditions suitable for expression of HPV L1 or L1 and L2 protein and assembly into a HPV VLP, and optionally isolating said VLP. In some examples, the cell is mammalian cell. In other examples of the methods the VSV is replication competent. In yet other examples, the VSV is replication-defective. In further examples, the VSV vector lacks G-protein function and is grown in an appropriate helper cell line.

As disclosed herein in the Examples, VSV HTLV-1 VLPs were purified by sucrose equilibrium gradient purification and VSV HPV VLPs were purified by cesium chloride equilibrium gradient purification. HTLV-1 VLPs and HPV VLPs can be purified by any means known to one of skill in the art. For example, HTLV-1 or HPV VLPs can be purified on sucrose gradients by the following method. BHK cells are infected at an m.o.i of 0.1 for 18 hours and then lysed in 50 mM Tris-HCL, pH 7.5, 50 mM NaCl, 0.1% NP-40, 1 mM PMSF, 10 μg/mg aprotinin, 10 μg/mg leupeptin, and 0.5 mM EDTA. The lysates are clarified by centrifugation through 30% sucrose for 6 hour at 150,000 xg. The resulting pellets are layered onto a continuous 30-70% sucrose gradient. One mL fractions are collected, centrifuged, an analyzed by SDS-Page and immunoblotting using antibody to VSV or HTLV-1 or HPV.

Methods of using recombinant VSV vectors of the invention

The subject VSV vectors and VSV produced VLPs can be used for a wide variety of purposes, which will vary with the desired or intended result. Accordingly, the present invention includes methods using the VSV vectors or VSV produced VLPs, such as for example HPV VLPs comprising L1 or L1 and L2 from any strain or HPV L1 and other viral proteins such as for example, combinations of different strains of HPV L1s such as for example, HPV 16 L1 and HPV 18 L1 proteins and for example, HTLV-1 VLPs comprising gag and env and compositions comprising the VSV vectors or VSV produced VLPs. In some examples, the composition further comprises a pharmaceutical excipient.

The present invention encompasses the use of the VSV vectors of the present invention and/or the VSV produced VLPs of the present invention for vaccine purposes to elicit an immune response when administered to an individual in need or to prevent infection; for post-vaccination purposes, that is for therapeutic purposes; to stimulate an immune response to an already established infection or to stimulate an immune response in an individual at risk for infection; for ex-vivo therapeutic approaches, i.e., to use VSV to express foreign proteins in transfected/infected antigen presenting cells such as dendritic cells and macrophages, for re-administration into potential patients, to stimulate an immune response to prevent infection or enhance a beneficial immune response in an individual already infected; and to use VSV produced VLPs to transfect antigen presenting cells for vaccine or therapeutic purposes.

In some examples, a VSV produced VLP, such as a HTLV-1 VLP or HPV VLP is administered in combination with a VSV vector or VSV particles. In some embodiments, the VSV vector may express an immunomodulatory gene, such as an interleukin, including interleukin 2 or 12; or an interferon, such as interferon-beta; or a chemokine or chemoattractant. In some examples, the VSV vectors or particles or VLPs are administered in combination with other treatment modalities.

VSV produced VLPs may be used to transfect antigen presenting cells for vaccine or therapeutic purposes. Dendritic cells (DC) are professional antigen presenting cells that are highly effective adjuvants for immunizing against pathogens and tumor antigens. Without being bound by theory, the potential merit of genetic approaches to loading DCs with antigens is to express high and sustained levels of proteins that can be subsequently processed and presented to T-lymphocytes. As shown herein recombinant VSV constructs that express recombinant green fluorescent protein (GFP) were produced and shown to efficiently transduce human and mouse dendritic cells and express the GFP to high levels. Accordingly, rVSV constructs that express HTLV-1 or HPV VLPs are used to transduce human dendritic cells ex vivo or in vivo.

In order to launch an immune response, dendritic cells (DC) must capture and process an antigen(s) in the periphery and present it to the rare antigen-specific T cells, which they encounter after migration to lymphoid organs. DC are able to sample, engulf and digest antigens of very diverse origin (viruses, bacteria, self proteins) and present them at the cell surface as short peptides in the context of MHC class I and II molecules. DC posses an arsenal of powerful co stimulatory molecules (CD40, CD80, CD86, DC-SIGN) and the potential to produce critical cytokines (chemokines, IL-12, etc), thus ensuring the initiation and the fate of acquired immunity.

A highly dynamic process is triggered by encountering antigens, while these are carried and processed to be appropriately presented, DC in turn experience a variety of changes: migratory, phenotypical, functional; all encompassed in the term of maturation. Essentially, DC maturation refers to a change from an antigen capturing to an antigen-presenting, T-cell priming mode, a process whereby DC convert antigens into efficacious immunogens, express the necessary cytokines and co-stimulatory molecules, thus appropriately initiating the specific, acquired clonal immunity.

DCs can be generated in vitro from cultures of human monocytes. Methods of producing dendritic cells are described in for example, WO 97/29182. As disclosed in WO 97/29182, mature dendritic cells are produced in vivo or in vitro from immature dendritic cells derived from PBMC pluripotential cells by contacting the immature dendritic cells with a dendritic cell maturation factor, such as a cytokine. Transduction of these DC with viral or tumor associated antigens, such as HTLV-1 or HPV antigens/VLPs leads to the presentation of these antigens via MHC class I molecules.

Another approach to antigen delivery is pulsing DC with antigenic peptides or proteins. This method has shown to induce antigen-specific CTL and to protect animals against subsequent viral or tumor challenge. Immunization with peptide and/or tumor lysate pulsed dendritic cells has also been reported in humans. Peptide loading strategy has several disadvantages, including HLA restriction and the fact that only a limited number of tumor specific peptides are known.

Recombinant VSV can transduce dendritic cells and express the foreign proteins, such as HTLV-1 or HPV VLPs to high levels. Thus, VSV could be used to load DCs with, potentially, multiple antigens and immunomodulatory genes for use in CTL stimulation against pathogens and tumor-mediated disease.

Methods of administration

Many methods may be used to administer or introduce the VSV vectors or VSV-viral particles or VLPs into individuals, including but not limited to, oral, intradermal, intramuscular, intraperitoneal, intravenous, intratumor, subcutaneous, and intranasal routes. The individual to which a VSV vector or viral particle or VLP is administered is a primate, or in other examples, a mammal, or in other examples, a human, but can also be a non-human mammal including but not limited to cows, horses, sheep, pigs, fowl, cats, dogs, hamsters, mice and rats. The present invention encompasses compositions comprising a VSV vector, a VSV viral particle or VSV produced VLPs wherein said compositions can further comprise a pharmaceutically acceptable carrier. The amount of VLP to be administered will depend on several factors, such as route of administration, the condition of the individual, the degree of aggressiveness of the malignancy (for cancer applications), and the particular VLP employed. Also, the VSV vector or VSV produced VLP may be used in conjunction with other treatment modalities, such as administration of interferon.

VSV vectors of the present invention comprising nucleic acid encoding HTLV-1 or HPV viral proteins or VSV viral particles or VSV produced HTLV-1 VLPs or HPV VLPs find use in immunogenic compositions, to elicit an immune response in an individual subject to or at risk for HTLV-1 associated disease or HPV associated disease, respectively. Whether VSV vector, VSV viral particle or VSV produced HTLV-1 VLPs or HPV VLPs is effective in eliciting an immunoprotective immune response can be determined by administering the subject VSV vector, VSV viral particle or VSV produced HTLV-1 VLP or HPV VLP to a test animal and after a period of time, challenging the animal with the HTLV-1 or HPV virus. Accordingly, the present invention provides vaccine compositions comprising VSV vector(s), VSV viral particle(s) or VSV produced HTLV-1 VLP(s) or HPV VLP(s) of the present invention and methods of eliciting an immune response comprising administering a vaccine composition to an individual subject to or at risk for a HTLV-1 or HPV associated disease. The present invention also provides methods for treating or ameliorating the symptoms associated with a HTLV-1 or HPV associated disease comprising administering to an individual subject to or at risk for a HTLV-1 or HPV associated disease a VSV vector(s), VSV viral particle(s) or VSV produced HTLV-1 VLP(s) or HPV VLP(s) of the present invention. An amount of a VSV vector(s), VSV viral particle(s) or VSV produced HTLV-1 VLP(s) or HPV VLP(s) effective to ameliorate a symptom associated with a HTLV-1 or HPV associated disease is present in a final concentration sufficient for amelioration of injury(ies) and includes, but is not limited to, a concentration which acts as a complete prophylaxis or treatment for a symptom. An effective amount to ameliorate a symptom can be administered in one or more administrations. An overview of vaccinology with special reference to papilloma virus vaccines is provided by Hilleman, 2000, J. Clin. Virol. Vol 19: 79-90.

Viral proteins/antigens used in the present invention can be either wild-type or recombinant polypeptides or fragments, thereof, or chemically synthesized.

The subject VSV vector, VSV viral particle or VSV produced HTLV-1 VLP or HPV VLP used in the present invention, can be conjugated to a vaccine carrier. Vaccine carriers are well known in the art: for example, bovine serum albumin (BSA), human serum albumin (HSA) and keyhole limpet hemocyanin (KLH).

A HTLV-1 or HPV VLP can be used in immunization or therapeutic treatments in conjunction with an adjuvant, or without an adjuvant. Examples of adjuvants include anti-CD40, antibodies, interferon, and LPS. In some examples, a VSV HTLV-1 or VSV HPV construct is administered alone. In other examples a VLP is administered alone. In some examples, a VSV-HPV vector construct or HTLV-1 vector construct of the present invention is administered followed by administration of a HPV VLP or HTLV-1 VLP, respectively, i.e., a VLP boost. In other examples, a VLP is administered followed by administration of a VSV vector construct. The precise dose of VSV vector or viral particles to be employed in the formulation will also depend on the route of administration, and the nature of the patient, and should be decided according to the judgment of the practitioner and each patient's circumstances according to standard clinical techniques. The exact amount of VSV vector or VLP utilized in a given preparation is not critical, provided that the minimum amount necessary to elicit an immune response is given. A dosage range of as little as about 10 μg, up to about a milligram or more, is contemplated.

Administrations are typically given periodically, while monitoring any response. Administration can be given, for example, subcutaneously, intratumorally, intravenously or intraperitoneally. A vaccine composition of the present invention can be administered using conventional devices including, but not limited to syringes, devices for intranasal administration, gene guns and vaccine guns.

Pharmaceutically acceptable carriers or excipients are well known in the art and include but are not limited to saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

In a specific embodiment, a lyophilized VSV vector, VSV viral particle or VLP of the invention is provided in a first container; a second container comprises diluent consisting of an aqueous solution of 50% glycerin, 0.25% phenol, and an antiseptic (e.g., 0.005% brilliant green).

The precise dose of VLP or VSV vector or viral particle to be employed in the formulation will also depend on the route of administration, and the nature of the patient, and should be decided according to the judgment of the practitioner and each patient's circumstances according to standard clinical techniques. The exact amount of VLP or VSV vector or particle utilized in a given preparation is not critical, provided that the minimum amount of VLP necessary to elicit an immune response is given.

The following examples are offered by way of illustration and should not be considered as limiting the scope of the invention.

### EXAMPLES

Materials and Methods

### Example 1

Generation of recombinant viruses, VSV-HPV-L1.

The HPV18 L1 coding sequences were PCR-amplified from an HPV18 cloned prototype of HPV18 DNA as template DNA. Unique restriction sites, XhoI and NheI (underlined), were incorporated into oligonucleotide primers as follows: 5′CCTTAACCTCGAGTCACTATGTGCCTGTATACACGG-3′ (sense) and 5′TCACTAGCTAGCTTACTTCCTGGCACGTACACGCAC-3′ (antisense). L1 PCR products were digested with XhoI and NheI and ligated into pVSV-XN2 which contained the entire VSV genome (M. J. Schnell et al. (1996, J Virol 70:2318-23). The final L1 construct, flanked by VSV transcription start and stop signals, was inserted between the VSV G and L gene products. Recombinant VSV expressing the L1 capsid protein was as previously described (M. Fernandez et al. (2002, J Virol 76:895-904). The recombinant vesicular stomatitis viruses were plaque purified and designated VSV-HPV 18 L1.

Growth Curves of recombinant viruses

BHK cells were infected with wild-type VSV, VSV-GFP, and VSV-HPV 18 L1 at an m.o.i. of 10. Supernatants from infected cells were obtained at the indicated times postinfection, and viral titers were determined by plaque assay.

Immunoflourescence

Expression of HPV 18 L1 capsid proteins was confirmed by immunoflourescence using a monoclonal antibody specific for HPV18 L1. Hela cells were infected with VSV-wild type, VSV-GFP, or VSV-HPV18 L1 at an m.o.i. of 10 for 5 hours and then fixed in 1% paraformaldehyde. The cells were incubated in 1:20 dilutions of primary antibody in 0.1% Brij-97/PBS for 2 hours at 4° C. and then incubated with FITC-conjugated goat anti-mouse (1:100; GIBCO-BRL) in 0.1% Brij-97/PBS for 1 hour at 4° C.

Western Immunoblotting

BHK cells were either mock-infected or infected at an m.o.i. of 1 with VSV-HPV18 L1, VSV-wild type, or VSV-GFP. After 18 hours, cell lysates were obtained and proteins electrophoretically separated on 10% polyacrylamide-SDS gels and transferred onto nitrocellulose membranes. After washing 3 times in (10% Tween/PBS) PBS-T buffer, the blots were incubated in primary HPV18-L1 antibody diluted 1:100 in 10% FBS/PBS (Research Diagnostics, Inc.). After washing in PBS-T buffer, the blots were incubated in secondary goat anti-mouse antibody for 2 h. Specific proteins were visualized using the enhanced chemiluminescence detection system (Pierce Chemicals; Rockford, Ill.).

CsCl gradient for isolation of HPV-like particles

Five 80% confluent, 15 cm dishes of Hela cells were infected with VSV-HPV-L1 at an m.o.i. of 5. Cells were harvested 16 hrs post-infection and washed once with PBS. The cell pellet was then resuspended in 2 ml cold PBS, frozen in liquid nitrogen, thawed and then brought to a final volume of 10 ml with cold PBS. The cells were then sonicated twice for 40 seconds each time. The cell lysate was clarified at 2,000 xg for 10 min and then layered onto 40% sucrose in PBS and centrifuged for 2.5 h at 110,000 xg. The resulting pellet was resuspended in PBS, then layered onto a 27% CsCl gradient and centrifuged for 20 h at 140,000 xg at 4° C. One ml fractions were then collected, diluted with PBS and centrifuged for 2 h at 110,000 xg. The even fractions were analyzed by SDS-PAGE and probed with antibodies against HPV-L1 (Research Diagnostics, Inc.) and VSV.

Generation of HPV-18 L1 expressing cell lines

The HPV-L1 encoding region was amplified by PCR and then cloned into the retroviral expression vector pFB-Neo (Stratagene; La Jolla, Calif.). The primers used for PCR amplification were

5′GAGCGAATTCAGTTATGTGCCTGTATACACAAATC3′ (sense) and 5′GCTTGCTCGAGTTACTTCCTGGCACGTACACGCAC3′ (antisense). The restriction sites EcoRI and XhoI were encoded into the primers respectively, and used for sub-cloning. VSV-G pseudotyped-MMLV based retroviruses encoding HPV-L1 were constructed according to the Vpack protocol (Stratagene). Balb/c derived TS/A cells were infected with HPV-L1 retrovirus and then selected for with neomycin. Single cell clones were screened and selected for HPV-L1 expression.

HPV-L1 ELISA

ELISAs for the generation of L1 specific antibody by vaccinated mice was conducted using lysates from 293T cells overexpressing HPV-L1 to coat 96 well plates. Different dilutions of mouse serum from vaccinated animals were incubated for 2 hrs followed by goat anti-mouse secondary antibody conjugated to horseradish peroxidase. The ELISAs were developed with TMB substrate (Pharmingen) and then read at 450 and 570 nm.

IFN-γ ELISPOT assays

IFN-γ ELISPOT assays were performed as previously described (B. Adkins et al. J Immunol 166:918-25) Splenocytes from vaccinated animals were incubated with TS/A cells stably expressing HPV-L1 for in vitro activation of T cells.

### Example 2

Generation of rVSV expressing HPV L1.

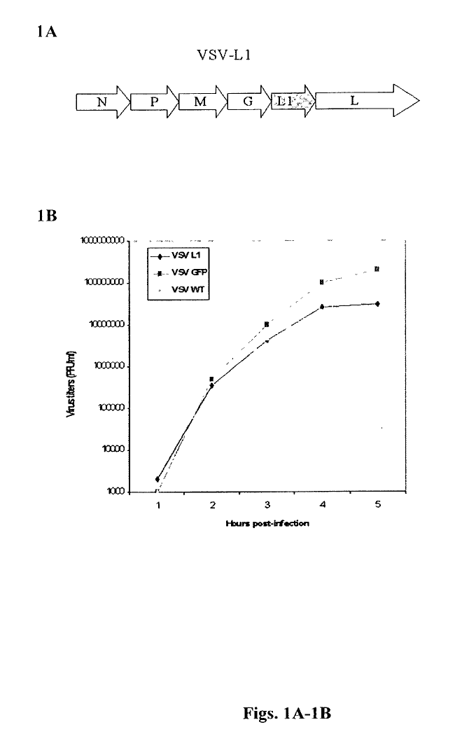

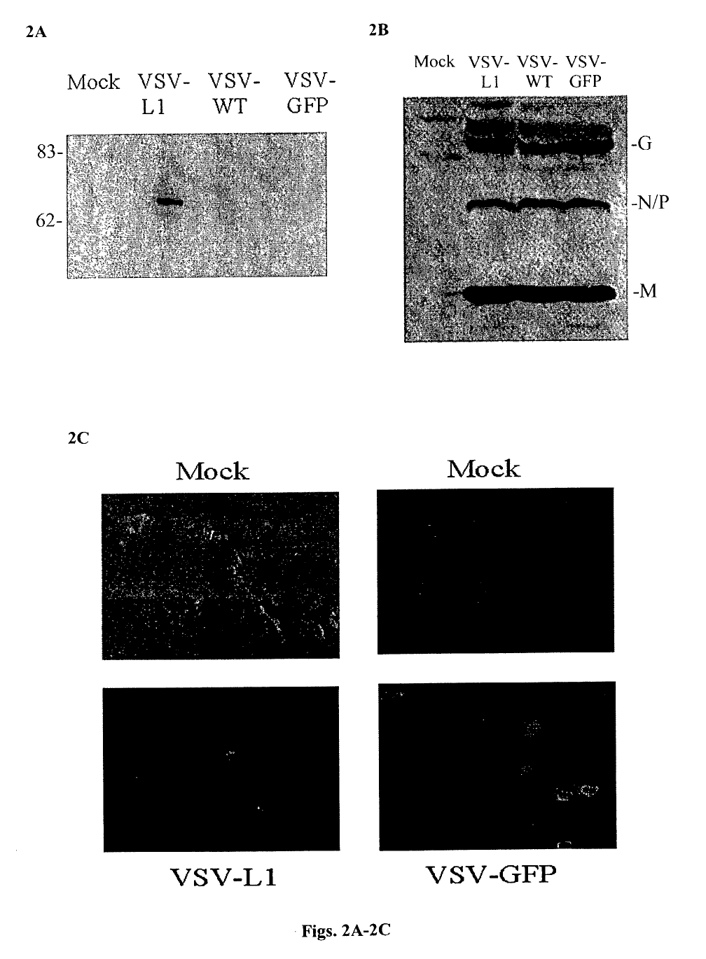

To evaluate whether recombinant (r) VSV could be utilized for the potential development of HPV-related vaccines and immunotherapies, the major HPV capsid protein L1 (strain 18), was cloned into a cDNA representing the VSV genome (pVSV-XN2; FIG. 1A; (M. J. Schnell et al. (1996. J Virol 70:2318-23)). To obtain recombinant VSV, the resultant plasmid (pVSV-HPV-L1) was transfected into BHK cells with VSV N, P and L genes and virus recovered (M. Fernandez et al. (2002, J. Virol 76:895-904). Viable recombinant VSV containing the coding region of the HPV structural proteins (referred to as VSV-HPV-L1) was plaque purified and exhibited similar growth properties to wild type VSV or recombinant VSV expressing green fluorescent protein (VSV-GFP) when examined by one-step growth curve analysis at a starting multiplicity of infection (m.o.i.) of 10 (FIG. 1B). To determine whether the recovered rVSV expressed the HPV L1 protein, BHK cells were infected with VSV-XN2 or VSV-HPV-L1 (m.o.i. of 1) and analyzed for L1 expression by western blot. FIG. 2A indicates that VSV-HPV-L1, but not VSV-XN2 infected cells, efficiently expressed the HPV-L1 protein. L1 was predominantly detected as a correctly sized protein of approximately 64 kDa (FIG. 2A). Expression of the VSV proteins was confirmed by western blot analysis using a polyclonal mouse antiserum to VSV (FIG. 2B). Confirmation of high-level HPV gene expression was achieved by immunofluorescent analysis of Hela cells infected with control VSV-XN2 or VSV-HPV-L1 (FIG. 2C). Antibody raised to HPV L1 strongly reacted to the nuclear and cytoplasmic regions of the cell, as previously reported for L1 localization in mammalian cells (P. Heino et al. (1995, Virology 214:349-59). Collectively, our data would indicate that VSV can efficiently express HPV L1 in human cells, which are likely posttranslationally processed in an authentic manner, having been synthesized in mammalian cells.

### Example 3

Generation and characterization of HPV-like particles (HPV-LPs).

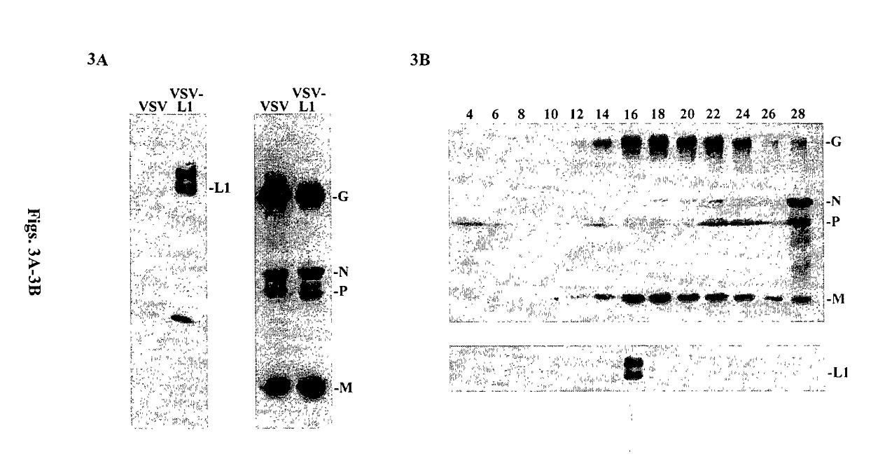

Evidence indicated that viable VSV was effectively generated to express at high levels, HPV structural protein L1. To further study HPV expression and association in mammalian cells using VSV, tissue culture medium of Hela cells infected with VSV or VSV-HPV-L1 was analyzed by western blot. L1 protein was detected in the cell medium indicating that a proportion of HPV capsid protein was being released from the cell, perhaps as a result of cytolysis (FIG. 3A). To clarify the association of HPV proteins with VSV, medium from VSV-XN2 or VSV-HPV-L1 infected cells was immunoprecipitated using a sheep antibody to VSV G. Following washing, complexes were separated by SDS-PAGE and immunoblotted against mouse antiserum raised to VSV. We found that the VSV structural proteins, N, P and M could be co-immunoprecipitated using the anti-G antibody. However, re-probing the blot with mouse anti-L1 antibody did not reveal L1 protein. Thus, L1 probably does not constitute a physical component of the VSV-HPV-L1 virion. This is most likely due to the HPV L1 lacking C-terminal regions of VSV G critically required for incorporation into VSV particles as they dissociate from the cell membrane (M. A. Whitt et al. (1989, J Virol 63:3569-78).

As indicated by our earlier immunofluorescence and immunoblot studies, HPV structural protein, L1, was predominantly found in the cell lysate fraction rather than the medium. To explore the association of intracellular HPV and VSV proteins, BHK cells were infected at an m.o.i. of 10 with VSV-XN2 or VSV-HPV-L1. Four hours post-infection, cells were labeled with ³⁵S-methionine/cysteine for another 12 hrs before being lysed. Cell extracts precipitated with a mouse anti-L1 mAb confirmed the presence of L1 alone, but not with any VSV proteins. Reciprocal co-immunoprecipitation studies using mouse antiserum to VSV also indicated little or no association of HPV L1 with VSV products, again indicating that HPV proteins are not strongly coupled with VSV complexes.

Previous studies have indicated that cooperative expression of L1 in bacteria and animal cells resulted in reassembly of the structural protein to form HPV-like particles (X. S. Chen et al. (2001, J Mol Biol 307:173-82); P. Heino et al. (1995, Virology 214:349-59); R. Kimbauer et al. (1993, J Virol 67:6929-36); V. Revaz et al. (2001, Virology 279:354-60). To further evaluate the association of the HPV L1 protein in our system, cell lysates previously infected with VSV-HPV-L1 or control VSV-XN2 were clarified and centrifuged through a cesium chloride equilibrium gradient. One ml fractions were collected from the bottom and analyzed by immunoblot for HPV L1 protein. This study revealed that L1 collectively sedimented in fraction 16, strongly suggesting the association of the capsid protein into virus-like particles (FIG. 3B). Similar findings were obtained upon sucrose gradient analysis of medium from VSV-HPV-L1 infected cells, indicating that some HPV complexes are also released from the cell. In contrast, VSV proteins N, P, M and G were found to reside throughout fractions 14-24.

In addition to these studies, uninfected Hela cells or Hela cells infected with either VSV-XN2 or VSV-HPV-L1 were analyzed by transmission electron microscopy (TEM) for the potential identification of HPV-L1 VLPs. When cells infected with VSV-HPV-L1, but not VSV-XN2, were examined by TEM, putative HPV-VLPs were found in the nucleus and appeared 50-60 nm in dimension. The apparent size would correlate with previous studies analyzing HPV which have calculated L1 VLPs to be approximately 50 nm in diameter (X. S. Chen et al. (2001, J Mol Biol 307:173-82); P. Heino et al. (1995, Virology 214:349-59); R. Kimbauer et al. (1993, J Virol 67:6929-36). HPV-LPs morphologically appeared to be icosahedral with a dense core, while VSV was observed to be characteristically bullet shaped or consists of a dense outer ring with a transparent core in cross-section analysis (J. J. Holland, (1987, The Rhabdoviruses Plenum, New York, p. 297-360); T. Nakai et al (1968, Virology 35:268-81).

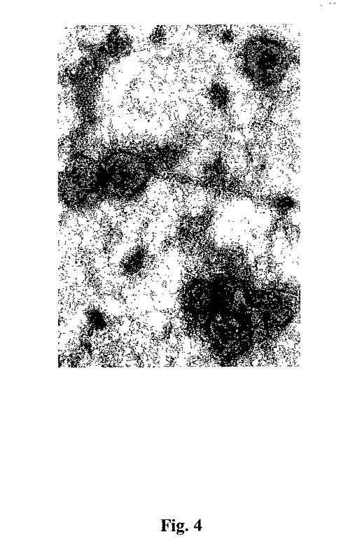

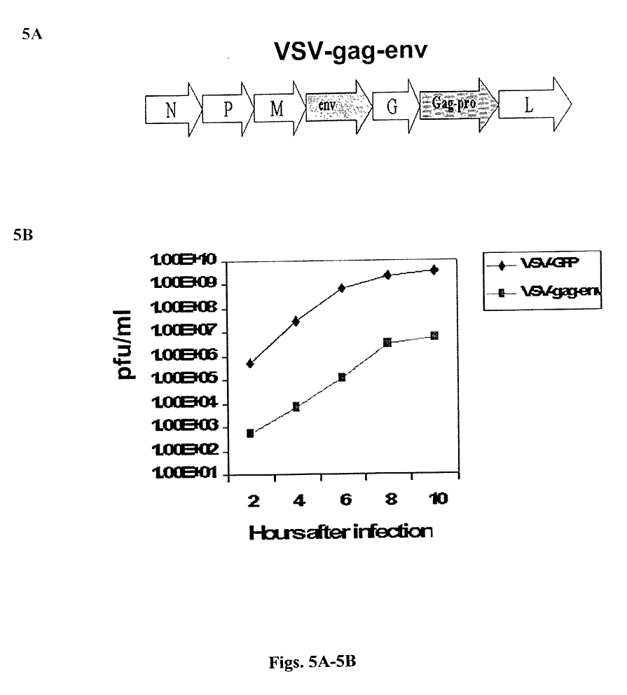

To complement TEM of VSV-HPV-L1 or control virus infected cells, centrifuged partially purified HPV-LPs were also examined by electron microscopy. Sucrose purified lysates from VSV-HPV-L1 Infected cells, but not from control rVSV infected cells appeared to contain HPV like particles with similar morphology to HPV observed in previous reports (FIG. 4). Collectively, these data would indicate that VSV can efficiently express HPV structural proteins in mammalian cells which may assemble into HPV-like particles.

### Example 4

Immunogenicity of VSV expressing HPV-L1.

Previous vaccine studies involving recombinant VSV expressing foreign viral antigens have demonstrated potent immune responses to the heterologous product in immunized animals (J. S. Kahn et al. (2001, J Virol 75:11079-87); A. Roberts et al. (1998, J Virol 72:4704-11); N. F. Rose et al. (2001, Cell 106:539-49); B. Schlereth et al. (2000, J Virol 74:4652-7). In light of these observations, we preliminarily examined in mice, whether VSV-HPV-L1 could induce an immune response to the HPV structural protein. Balb/c mice (6-8 weeks) were intravenously (i.v.) injected with 2.5×10⁶ pfu of VSV-GFP or VSV-HPV-L1 or phosphate buffered saline (PBS), followed by a second inoculation (5×10⁶ pfu, i.v) two weeks later. Since antibody to L1 has been shown to be critical for neutralization of infectious HPV we first examined the potential generation of L1 antibodies using enzyme-linked immunosorbent assay (ELISA). Analysis of serum 21 days post initial inoculation indicate that mice vaccinated with VSV-HPV-L1, generated a good humoral response to the HPV-L1 protein. Collectively, these findings would indicate that VSV-HPV-L1 is an efficient vehicle to generate antibodies to HPV L1.

The generation of a multispecific cytotoxic T cell (CTL) response is also considered to be important for the clearance of HPV during acute infections in humans. Therefore, to determine if VSV-HPV-L1 was able to generate CD8⁺ T cell responses to the structural proteins of HPV, IFN-γ ELISPOT assays were performed on splenocytes isolated from PBS, VSV-GFP, or VSV-HPV-L1 four weeks following the initial vaccination as previously described. Only CTLs from VSV-HPV-L1 vaccinated mice were activated by the HPV-L1 expressing TS/A cells as demonstrated by the production of IFN-γ. These data would indicate that intravenously administered VSV-HPV-L1 is able to not only induce humoral activity to HPV proteins, but also stimulate CTL activity to the capsid protein of HPV.

As route of inoculation can affect both the strength and type of immunity generated, we also examined the primary response generated by intraperitoneal (i.p.) inoculation of 5×10⁷ pfu of VSV-GFP, VSV-HPV-L1, or PBS. By day 28, significant anti-L1 antibody levels were detected in VSV-HPV-L1 immunized mice. Furthermore, 7 days after receiving VSV-HPV-L1, splenocytes from vaccinated animals were analyzed for IFN-γ production following stimulation with HPV-L1 expressing cells. Our data indicate that only VSV-HPV-L1 infected mice contained evidence of cytotoxic T-cells specific to the L1 protein following vaccination.

### Example 5

Construction of VSV-HTLV-1 Gag Env

To evaluate the potential of a VSV-based vaccine for preventing HTLV-1 infection, the Gag-Pro and Env genes of HTLV-1 were cloned into the plasmid pVSV-XN2 (FIG. 5A). The Gag-Pro and Env inserts were amplified from pCMV Gag and pHT-Env-pX-CMV plasmids (from Dr. David Deerse) respectively by PCR. For Gag-Pro, the forward and reverse primers were 5′-CGGCATGTCGACCACTATGGGCCAAATCTTTTCCCGT and 5′-CGCTGTCTAGATTAGAGAGTTAGTGGCCCGCAGGT respectively.

The Gag-Pro PCR Product was digested with Sal I and Xba I and ligated to pVSV-XN2 that had been digested with XhoI (compatible with Sal I) and NheI (compatible with XbaI).

PCR for Env was carried out using the following upstream and downstream primers: 5′GCGGACTAGTCACTATGGGTAAGTTTCTCGCCACT and

5° CATATCTTGCGGCCGCTTACAGGGATGACTCAGGGTT respectively.

The PCR Product was digested with SpeI and NotI and cloned into the SpeI, NotI sites of VSV-Gag-Pro between the M and G genes.

The procedure for recovering infectious recombinant VSV viruses was similar to that described previously. Recombinant VSVs expressing these genes were recovered in cells expressing the full-length anti-genomic RNA containing the additional genes as well as the nucleocapsid, phosphoprotein, and polymerase proteins.

Virus growth in vitro

The growth of recombinant viruses was analyzed by one step growth analysis. BHK cells were infected with VSV-GFP or VSV-Gag-Env at a multiplicity of infection (m.o.i.) of 10 PFU per cell. The culture supernatants were harvested at the indicated times and subjected to titer determination by a standard plaque assay on BHK-21 cells. The results showed that the recombinant viruses were viable but attenuated in their replication (FIG. 5B).

Western blot analysis

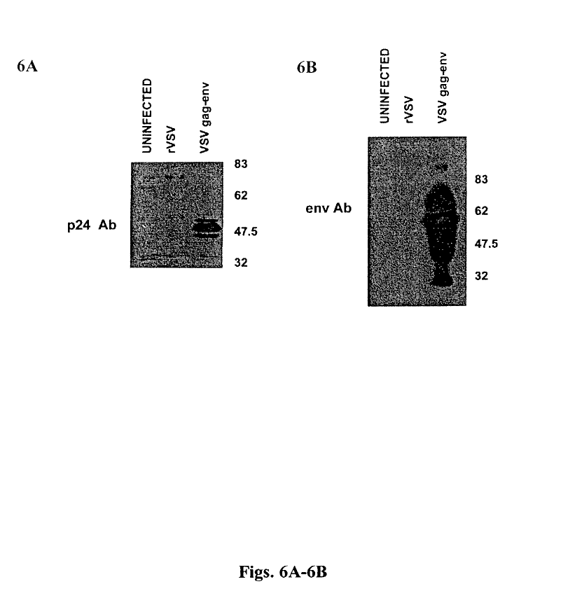

To determine whether the recovered rVSV expressed HTLV-1 proteins, BHK cells were either mock-infected or infected at an m.o.i. of 1 with VSV-Gag-Env or VSV-GFP for 24 hours. Cell lysates were harvested and the proteins electrophoresed on 10% polyacrylamide-SDS gels. Following transfer onto nitrocellulose membranes and the blot was incubated overnight with anti-Env or anti-p24 antibody followed by a secondary goat anti-mouse antibody for 2 h. Proteins were visualized using the enhanced chemiluminescence detection system. FIG. 6 indicates that VSV-gag-env, but not VSV-GFP infected cell lysates, efficiently expressed the HTLV-1 structural proteins.

Immunofluorescence.

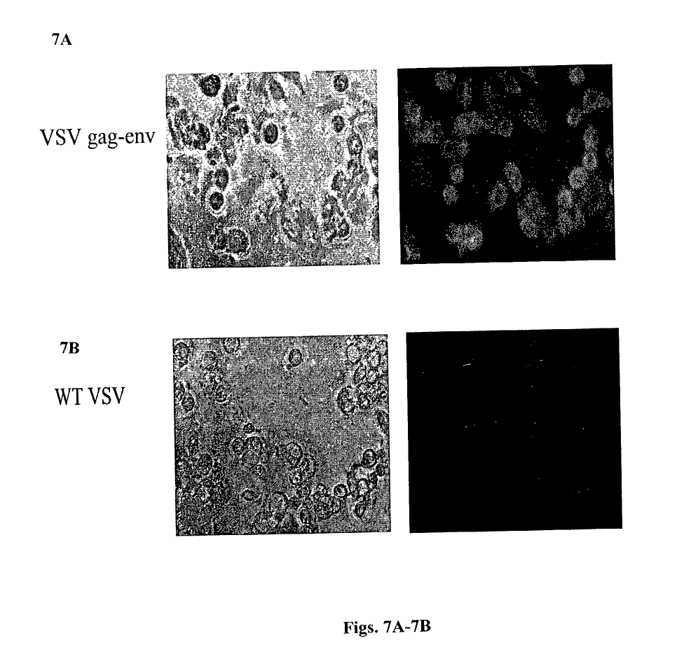

Expression of the Env protein was confirmed by immunofluorescence using an anti-env antibody. BHK cells were grown on coverslips and then infected with wild type VSV or VSV-Gag-Env at an m.o.i. of 1 for 16 h. They were then washed in PBS and fixed in 1% paraformaldehyde for 30 min. The cells were incubated with 1:50 dilutions of anti-Env antibody for 2 h at 4° C., washed with PBS/200 mM glycine, and then incubated with FITC-conjugated goat anti-mouse (1:100; Gibco-BRL;) in 0.1% Brij-97/PBS for 1 h at 4° C. Immunostained cells were washed three times in PBS and treated with Slowfade Anti-Fade kit (Molecular Probes; Eugene, Oreg.). As shown in FIG. 7, there is abundant expression of Env in VSV-Gag-Env infected cells.

In conclusion, recombinant VSV with the HTLV-1 gag and env genes were generated that expressed high levels of the heterologous proteins.

Immunoblot analysis of cell medium

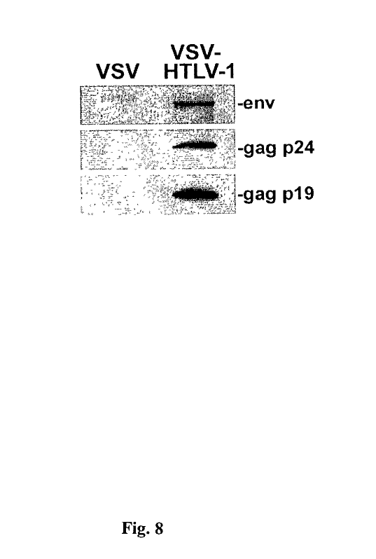

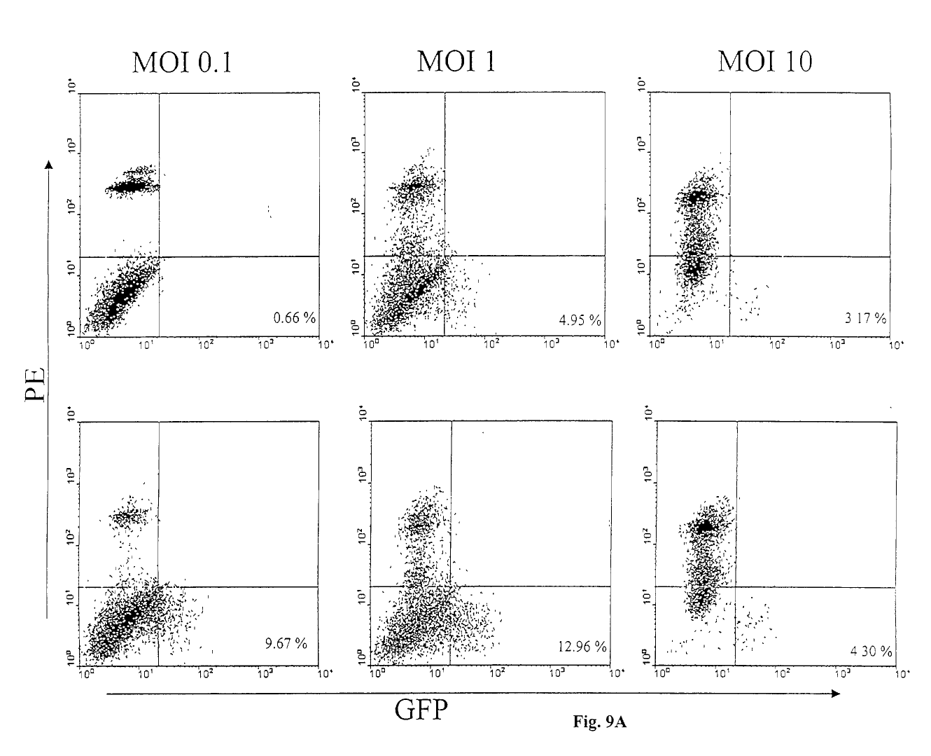

Approximately 1×10⁷ BHK cells were infected with either VSV-XN2 or VSV-HTLV1-gag/env at an m.o.i. of 0.01 for 24 hours. Cell medium was harvested and clarified of cell debris by centrifugation at 2,000 rpm for 5 minutes. The cell medium was the centrifuged through a 10% sucrose cushion in PBS for one hour at 110,000 x g at 4° C. The resulting pellets were then resuspended in cold PBS. Equal amounts of protein from the cell medium of VSV-XN2 and VSV-HTLV1 -gag/env were analyzed by immunoblot analysis. HTLV-1 proteins were detected using monoclonal antibodies to env, gag-p24, and gag-p19 (Cell Sciences, Norwood, Mass.) followed by horseradish peroxidase conjugated goat anti-mouse antibody. See FIG. 8.

### Example 6

Purification of HTLV-1 like particles by equilibrium gradient purification. 6×10⁷ BHK cells were infected with VSV-HTLV 1 -gag/env at a multiplicity of infection of one. 24 hours after infection, the cell medium was collected and clarified of cellular debris by low speed centrifugation at 2,000 rpm for 5 minutes. Clarified medium was then centrifuged through 10% sucrose (in PBS) for 2 hours at 110,000 x g at 4° C. The resulting pellets were resuspended in 10 mM Tris pH 7.5/100 mM NaCl and then layered onto a continuous 20-70% sucrose equilibrium gradient. The gradient was centrifuged for 18 hours at 110,000 x g and 4° C. One milliliter fractions were collected from the bottom, diluted in 10 mM Tris/100 mM NaCl and then spun for 1 hour at 110,000 x g. HTLV-1 particles were suspended in Tris/NaCl and analyzed by immunoblot analysis for expression of gag and env.

### Example 7

VSV from cDNA

A cDNA clone representing the entire 11,161 nucleotides of VSV was generated and unique Xho I/Nhe I sites were added to facilitate entry of a heterologous gene. Transcription of the cDNA is dependent on T7 RNA polymerase. Vaccinia vTF7-3 is used to infect baby hamster kidney cells (BHK-21), to provide a source of polymerase. Subsequently, VSV cDNA is transfected into the same cells 20 together with three other plasmids that express the VSV N, P and L proteins. These latter three proteins facilitate the assembly of nascent VSV antigenomic RNA into nucleocapsids and initiate the VSV infectious cycle. After 24 hours, host cells are lysed, clarified and residual vaccinia removed by filtration through a 0.2 um filter onto fresh BHK cells. Only recombinant VSVs are produced by this method since no wild-type VSV can be generated.

### Example 8

Characterization of recombinant VSVs (rVSVs).

Cells infected with rVSV or wild-type VSV are metabolically labeled with [³⁵S]methionine. Cells are lysed and aliquots analyzed by SDS-PAGE. Since VSV inhibits host proteins synthesis, only viral proteins are made, including heterologous genes inserted into its genome. Cells infected with rVSVs will have an extra protein (i.e. HPV or HTLV-1 protein) being synthesized compared to control cells infected with VSV alone. VSV mRNAs are detected by a similar manner using radiolabeled dUTP. In many cases, antibody to the heterologous protein exists or are prepared by one of skill in the art. Therefore, ELISAs are used to detect the expression of heterologous proteins, such as, HPV L1 or HTLV-1 gag and env. High levels of heterologous protein expression have been obtained in all recombinant systems examined.

### Example 9

Growth of VSV

Large amounts of VSV (Indiana strain) and recombinant VSV are purified by sucrose gradients. Essentially, BHK cells are infected at 0.01 m.o.i and after 24 hours, where >80% of cells usually exhibit CPE/apoptosis, supernatants are collected and clarified by centrifugation. Clarified supernatants are purified by centrifugation through 10% sucrose and the viral pellets resuspended and layered onto continuous 35-55% sucrose gradients. The gradients are centrifuged at 10,000 g for 18 hours at 4° C. and virus retrieved and pelleted by further centrifugation and 15,000 rpm at 4° C. for 1 hour. Viruses are resuspended in PBS, concentrations determined by standard plaque assays and stored in aliquots at −80° C. (30).

### Example 10

### Generation of replication-defective recombinant VSV.

VSV that lacks the G protein function and which express HPV or HTLV-1 structural proteins are constructed. Such viruses are generated in helper cells (CHO) that have been constructed to inducibly express the VSV G protein. Following infection of target cells, resultant viruses infect cells because they contain the VSV G from the helper cell. However, following infection and replication, progeny viruses will lack the receptor G and cannot disseminate to infect surrounding tissue. rVSV lacking part or all of M and N protein function are produced. Such viruses are generated in helper cells (CHO) that have been constructed to inducibly express the VSV regions lacking in the rVSV construct.

### Example 11

Generation of Dendritic Cells

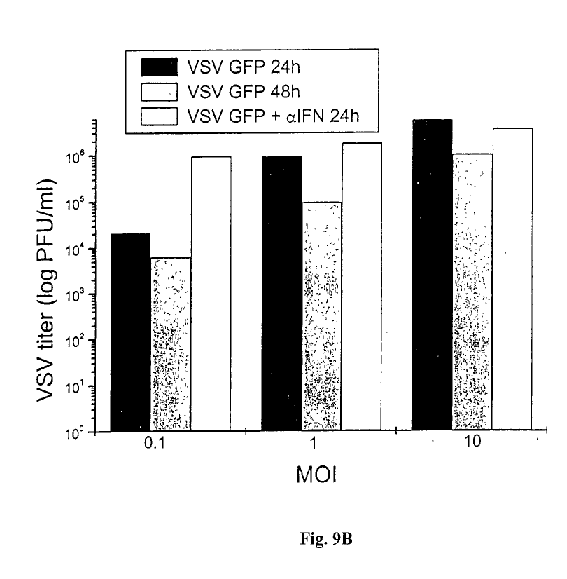

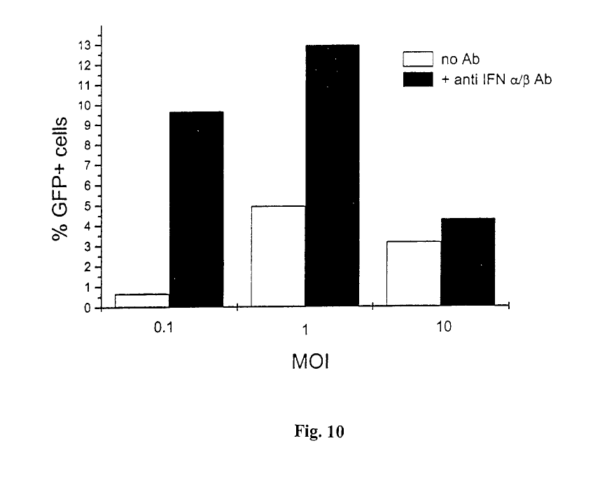

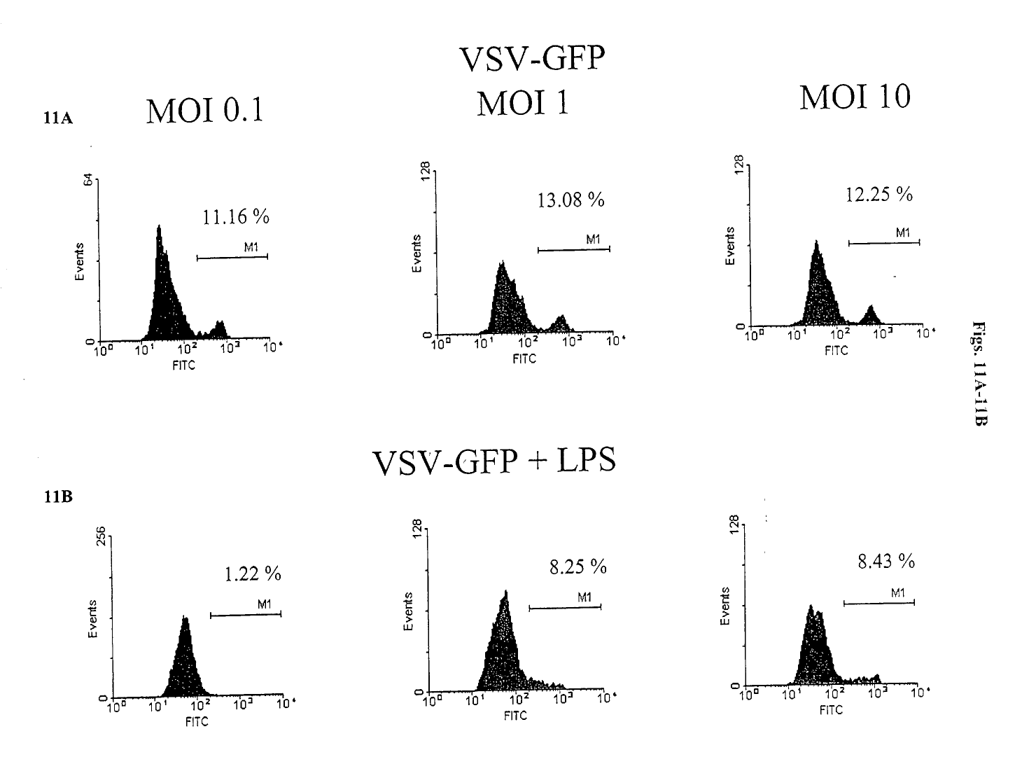

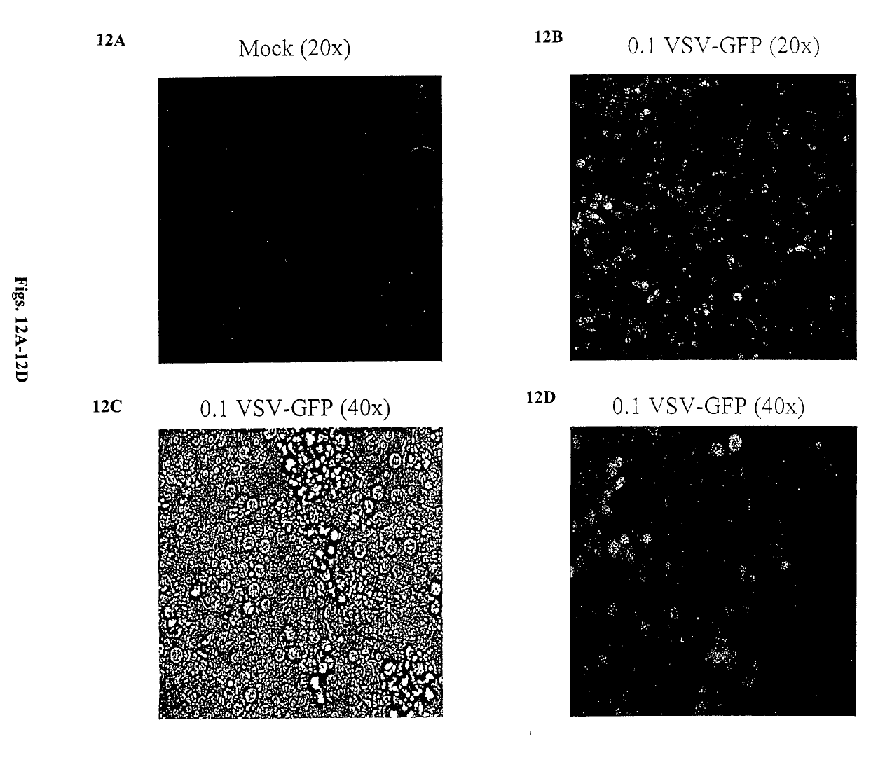

Bone marrow was flushed from the long bones of the hind limbs of Balb/c mice. Upon depletion of erythrocytes, the cells were cultures in RPMI 1640 supplemented with 10% FBS, penicillin-streptomicin and 500 U/ml recombinant murine GM-CSF and 500 U/ml recombinant murine IL-4 (both from Sigma-Aldrich). On day 2 of culturing, supernatant was removed and replenished with fresh medium and cytokines. On day 6, non adherent cells were collected and further purified with CD11c MACS micro beads (Miltenyi Biotech, Auburn, Calif.). For phenotypic analysis the expression of the cell surface markers CD11c, CD80, CD86, MHC class II and CD40 was tested using commercially available mAbs (all from PharMingen). As shown in FIGS. 9A-9B, DCs were infected for 1 hr with VSV virus coding for the GFP protein at different m.o.i.s and cells analyzed by FACS 24 hours later. The percentage of live and GFP+ cells is shown in the lower right quadrant. Lower panel represents FACS analysis of DC cultured in the presence of anti mouse IFN α/β antibodies (1000 U/ml) that were added to DC culture following virus infection. Upper panel represent DC cultures without addition of neutralizing IFN antibodies.

The present invention is not to be limited in scope by the specific embodiments described herein. Various modifications of the invention in addition to those described herein will become apparent to those skilled in the art from the foregoing description and accompanying figures. Such modifications are intended to fall within the scope of the appended claims.

1. A recombinant vesicular stomatitis virus (VSV) vector comprising isolated nucleic acid encoding part or all of a HTLV-1 Gag gene and part or all of a viral Env gene, wherein said part or all of said HTLV-1 Gag gene and said part or all of the viral Env gene are capable of assembling into a HTLV-1 virus like particle.

2. The recombinant VSV vector of claim 1 wherein said viral Env gene is a HTLV-1 Env gene.

3. The recombinant VSV vector of claim 1 wherein said VSV vector is replication competent.

4. The recombinant VSV vector of claim 1 wherein said VSV vector is replication-defective.

5. The recombinant VSV vector of claim 4 wherein said VSV vector lacks G-protein function.

6. An isolated nucleic acid encoding the recombinant VSV vector of claim 1.

7. A cell comprising the VSV vector of claim 1, and progeny thereof.

8. A HTLV-1 virus like particle comprising a HTLV-1 Gag gene and part or all of a viral Env gene.

9. The HTLV-1 virus like particle of claim 8 wherein said part or all of the viral Env gene is a HTLV-1 Env gene.

10. A composition comprising the recombinant VSV vector of claim 1.

11. A composition comprising the HTLV-1 virus like particle of claim 9.

12. The composition of claim 10 or 11 further comprising a pharmaceutically acceptable excipient.

13. A method of producing a HTLV-1 virus like particle (VLP) comprising growing a cell according to claim 7 under conditions suitable for expression of the HTLV-1 Gag and viral Env genes and assembly into a VLP and optionally isolating said VLP.

14. The method of claim 13 wherein said cell is mammalian cell.

15. The method of claim 13 wherein said VSV is replication competent.

16. The method of claim 13 wherein said VSV is replication-defective.

17. The method of claim 16 wherein said VSV vector lacks G-protein function.

18. A HTLV-1 virus like particle made by the method of claim 13.

19. A composition comprising the HTLV-1 particle of claim 18.

20. A method of eliciting an immune response in an individual comprising administering to the individual the HTLV-1 virus like particle of claim 9.

21. A method of eliciting an immune response in an individual comprising administering to the individual the HTLV-1 virus like particle made by the method of claim 13.

22. A vaccine composition comprising the HTLV-1 virus like particle of claim 9.

23. A vaccine composition comprising the HTLV-1 virus like particle made by the method of claim 13.

24. A recombinant vesicular stomatitis viral particle comprising the VSV vector of claim 1.

25. The composition of claim 19 wherein said HTLV-1 virus like particle is present in the composition in an amount effective to elicit an immune response in an individual.

26. A recombinant vesicular stomatitis virus (VSV) vector comprising isolated nucleic acid encoding a HPV L1 protein wherein said HPV L1 protein is capable of assembling into a HPV virus like particle.

27. The recombinant VSV vector of claim 26 wherein said VSV vector further comprises nucleic acid encoding HPV L2 protein.

28. The recombinant VSV vector of claim 26 wherein said VSV vector is replication competent.

29. The recombinant VSV vector of claim 26 wherein said VSV vector is replication-defective.

30. The recombinant VSV vector of claim 29 wherein said VSV vector lacks G-protein function.

31. An isolated nucleic acid encoding the recombinant VSV vector of claim 26.

32. A cell comprising the VSV vector of claim 26, and progeny thereof.

33. A composition comprising the recombinant VSV vector of claim 26.

34. A method of producing a HPV virus like particle (VLP) comprising growing a cell according to claim 32 under conditions suitable for expression of HPV L1 protein and assembly into a VLP and optionally isolating said VLP.

35. The method of claim 34 wherein said cell is mammalian cell.

36. The method of claim 34 wherein said VSV is replication competent.

37. The method of claim 34 wherein said VSV is replication-defective.

38. The method of claim 37 wherein said VSV vector lacks G-protein function.

39. A HPV virus like particle made by the method of claim 34.

40. A composition comprising the HTLV-1 particle of claim 39.

41. A method of eliciting an immune response in an individual comprising administering to the individual the HPV virus like particle made by the method of claim 34.

42. A vaccine composition comprising the HPV virus like particle made by the method of claim 26.

43. A recombinant vesicular stomatitis viral particle comprising the VSV vector of claim 26.

44. The recombinant VSV vector of claim 26 wherein said HPV is a HPV strain selected from the group consisting of HPV 16, HPV 18, HPV 31, HPV 33 or HPV 45.

45. The recombinant VSV vector of claim 1 comprising additional HTLV-1 viral proteins.

46. The recombinant VSV vector of claim 26 comprising additional HPV viral proteins.
